(19) United States
(12) Patent Application Publication
(10) Pub. No.: US 2022/0184604 A1
(43) Pub. Date: Jun. 16, 2022

(54) Methods, Devices and Systems for Enhanced Transduction Efficiency

(71) Applicants: Emory University, Atlanta, GA (US); Children's Healthcare of Atlanta, Inc., Atlanta, GA (US)

(72) Inventors: Wilbur A. Lam, Atlanta, GA (US); Reginald Tran, Atlanta, GA (US); Christopher B. Doering, Atlanta, GA (US); Harold Trent Spencer, Atlanta, GA (US)

(21) Appl. No.: 17/603,266

(22) PCT Filed: Apr. 13, 2020
(86) PCT No.: PCT/US2020/028006

§ 371 Date: Oct. 12, 2021

Related U.S. Application Data
(60) Provisional application No. 62/833,111, filed on Apr. 12, 2019.

(51) Int. Cl. B01L 3/00

(52) U.S. Cl. CPC B01L 3/502; B01L 2300/0861; B01L 2200/026; B01L 2200/025

(57) ABSTRACT

The systems, devices, and methods utilize devices configured to (i) control the loading of each channel layer and/or (ii) prevent formation of bubbles within the channels. A device may include two or more stacked layers. The two or more stacked layers may include a first layer and a second layer. The first entry region diameter of the first layer and the second entry region diameter of the second layer may be different; and/or the first exit region diameter of the first layer and the second exit region diameter of the second layer may be different; and/or one or more of the first channel dimensions (e.g., length and/or width) of the first layer and the one or more of the second channel dimensions (e.g., length and/or width) of the second layer may be different.

### CROSS-REFERENCE TO RELATED APPLICATIONS

This application claims the benefit of U.S. Provisional Application No. 62/833,111 filed Apr. 12, 2019. The entirety of this application is hereby incorporated by reference for all purposes.

### BACKGROUND

Manufacturing complexity and limited viral vector availability has generally hampered manufacturing capacity of novel gene and cell therapies, slowing widespread clinical implementation and commercialization.

### SUMMARY

Thus, there is need for more efficient systems, devices and methods that can more quickly and efficiently achieve therapeutic levels of transduction using less amount of the viral vector while accommodating clinically relevant cell numbers that can be as large as one billion cells or greater.

In some embodiments, the devices may include a device that includes two or more layers. The two or more stacked layers may include a first layer and a second layer. The first layer may include a first fluid path. The first fluid path may include a first entry region having a first entry region diameter, a first exit region having a first exit region diameter, and a first channel disposed between the first entry region and the first exit region. The first channel may have first channel dimensions. The first channel dimensions may include a first channel length and a first channel width.

In some embodiments, the second layer may include a second fluid path that may be parallel to the first fluid path and may be in fluid communication with the first fluid path. The second fluid path may include a second entry region having a second entry region diameter, a second exit region having a second exit region diameter, and a second channel disposed between the second entry region and the second exit region. The second channel may have second channel dimensions. The second channel dimensions may include a second channel length and a second channel width.

In some embodiments, the first entry region diameter and the second entry region diameter may be different; and/or the first exit region diameter and the second exit region diameter may be different; and/or one or more of the first channel dimensions and the one or more of the second channel dimensions may be different.

In some embodiments, the first entry region and the second entry region may be aligned with respect to its respective center and may be in fluid communication.

In some embodiments, the first exit region and the second exit region may be aligned with respect to its respective center and may be in fluid communication.

In some embodiments, the first exit region diameter may be larger than the second exit region diameter.

In some embodiments, the first entry region diameter may be smaller than the second entry region diameter.

In some embodiments, the device may further include a third layer. The third layer may include at least one loading region and/or at least one collection region. The first layer may be disposed between the third layer and the second layer.

In some embodiments, the at least one loading region of the third layer may be aligned with the first entry region and the second entry region with respect to its respective centers and may be in fluid communication.

In some embodiments, the third layer may include a fluid path, the fluid path may include the at least one collection outlet, an exit region and a channel may be disposed therebetween. The exit region of the third layer may be aligned with the first exit region and the second exit region with respect to its respective centers and may be in fluid communication.

In some embodiments, the at least one loading region of the third layer may be separate from the fluid path of the third layer.

Each layer may include at least one loading region. The at least one loading region of the first layer may be separate from the first fluid path. The at least one loading region of the second layer may be in fluid communication with the second fluid path. The at least one loading region of the first layer and the at least loading region of the second layer may be aligned with respect to its respective centers and may be in fluid communication.

In some embodiments, a device may include two or more layers. The two or more stacked layers may include a first layer and a second layer. The first layer may include a first fluid path. The first fluid path may include a first entry region having a first entry region diameter, a first exit region having a first exit region diameter, and a first channel disposed between the first entry region and the first exit region. The first channel may have first channel dimensions. The first channel dimensions may include a first channel length and a first channel width. The second layer may include a second fluid path that may be in fluid communication with the first fluid path. The second fluid path may include a second entry region having a second entry region diameter, a second exit region having a second exit region diameter, and second channel disposed between the second entry region and the second exit region. The second channel may have second channel dimensions. The second channel dimensions may include a second channel length and a second channel width. In some embodiments, the first exit region diameter and the second exit region diameter may be different.

In some embodiments, the first exit region diameter may be larger than the second exit region diameter.

In some embodiments, the first exit region and the second exit region may be aligned with respect to its respective center and may be in fluid communication. The first entry region diameter may be smaller than the second entry region diameter.

In some embodiments, the device may further include a third layer may include at least one loading region and/or at least one collection region. The first layer may be disposed between the third layer and the second layer. In some embodiments, at least one loading region of the third layer may be aligned with the first entry region and the second entry region with respect to its respective centers and may be in fluid communication.

In some embodiments, the third layer may include a fluid path. The fluid path may include the at least one collection region, an exit region, and a channel disposed therebetween. The exit region of the third layer may be aligned with the first exit region and the second exit region with respect to its respective centers and may be in fluid communication.

In some embodiments, the at least one loading region may be separate from the fluid path. The at least one loading region may be aligned with the first entry region and the second entry region with respect to its respective centers and may be in fluid communication.

In some embodiments, each layer may include at least one loading region. The at least one loading region of the first layer may be separate from the fluid communication with the first fluid path. The at least one loading region of the second layer may be in fluid communication with the second fluid path. The at least one loading region of the first region and the at least loading region of the second layer may be aligned with respect to its respective centers and may be in fluid communication.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, emphasis being placed upon illustrating the principles of the disclosure.

FIG. 1A shows top view of a device according to some embodiments;

FIG. 1B shows a cross-sectional view of the device of FIG. 1A;

FIG. 1C shows an exploded view showing a top view of each layer of the device of the FIG. 1A;

FIG. 1D shows an exploded cross-sectional view of each layer shown in FIG. 1C;

FIG. 2A shows top view of another device according to some embodiments;

FIG. 2B shows a cross-sectional view of the device of FIG. 2A;

FIG. 2C shows an exploded view showing a top view of each layer of the device of the FIG. 2A;

FIG. 2D shows an exploded cross-sectional view of each layer shown in FIG. 2C;

FIG. 3A shows top view of another device according to some embodiments;

FIG. 3B shows a cross-sectional view of the device of FIG. 3A;

FIG. 3C shows an exploded view showing a top view of each layer of the device of the FIG. 3A;

FIG. 3D shows an exploded cross-sectional view of each layer shown in FIG. 3C;

FIG. 4A shows top view of another device according to some embodiments;

FIG. 4B shows a cross-sectional view of the device of FIG. 4A;

FIG. 4C shows an exploded view showing a top view of each layer of the device of the FIG. 4A;

FIG. 4D shows an exploded cross-sectional view of each layer shown in FIG. 4C;

FIG. 5A shows top view of another device according to some embodiments;

FIG. 5B shows a cross-sectional view of the device of FIG. 5A;

FIG. 5C shows an exploded view showing a top view of each layer of the device of the FIG. 5A;

FIG. 5D shows an exploded cross-sectional view of each layer shown in FIG. 5C;

FIG. 6A shows top view of another device according to some embodiments;

FIG. 6B shows a cross-sectional view of the device of FIG. 6A;

FIG. 6C shows an exploded view showing a top view of each layer of the device of the FIG. 6A;

FIG. 6D shows an exploded cross-sectional view of each layer shown in FIG. 6C;

FIG. 7A shows top view of another device according to some embodiments;

FIG. 7B shows a cross-sectional view of the device of FIG. 7A;

FIG. 7C shows an exploded view showing a top view of each layer of the device of the FIG. 7A;

FIG. 7D shows an exploded cross-sectional view of each layer shown in FIG. 7C;

FIG. 8A shows top view of another device according to some embodiments;

FIG. 8B shows a cross-sectional view of the device of FIG. 8A;

FIG. 8C shows an exploded view showing a top view of each layer of the device of the FIG. 8A;

FIG. 8D shows an exploded cross-sectional view of each layer shown in FIG. 8C;

FIG. 9A shows top view of another device according to some embodiments;

FIG. 9B shows a cross-sectional view of the device of FIG. 9A;

FIG. 9C shows an exploded view showing a top view of each layer of the device of the FIG. 9A;

FIG. 9D shows an exploded cross-sectional view of each layer shown in FIG. 9C;

FIG. 10A shows top view of another device according to some embodiments;

FIG. 10B shows a cross-sectional view of the device of FIG. 10A;

FIG. 10C shows an exploded view showing a top view of each layer of the device of the FIG. 10A;

FIG. 10D shows an exploded cross-sectional view of each layer shown in FIG. 10C;

FIG. 11A shows top view of another device according to some embodiments;

FIG. 11B shows a cross-sectional view of the device of FIG. 11A;

FIG. 11C shows an exploded view showing a top view of each layer of the device of the FIG. 11A;

FIG. 11D shows an exploded cross-sectional view of each layer shown in FIG. 11C;

FIG. 12A shows top view of another device according to some embodiments;

FIG. 12B shows a cross-sectional view of the device of FIG. 12A;

FIG. 12C shows an exploded view showing a top view of each layer of the device of the FIG. 12A;

FIG. 12D shows an exploded cross-sectional view of each layer shown in FIG. 12C;

FIG. 13A shows top view of another device according to some embodiments;

FIG. 13B shows a cross-sectional view of the device of FIG. 13A;

FIG. 13C shows an exploded view showing a top view of each layer of the device of the FIG. 13A; and

FIG. 13D shows an exploded cross-sectional view of each layer shown in FIG. 13C.

### DESCRIPTION OF THE EMBODIMENTS

In the following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

The systems, methods, and devices according to the disclosure can address the manufacturing complexity and limited viral vector availability that has contributed to hampering manufacturing capacity of novel gene and cell therapies and slowing widespread clinical implementation and commercialization. The systems, methods, and devices may utilize a multi-layer stacked device that allows for scalability in clinical gene transfer. For example, the device may be capable of transducing billions of cells.

In some embodiments, the devices can include a plurality of layers of channels connected in parallel. The devices according to the disclosure can be scaled up while preserving the fluid channel height enabling one billion cells or more to be transduced similarly to a small scale device containing one million cells.

In some embodiments, the devices according to the disclosure can include features configured to (i) passively control the loading of each channel layer and/or (ii) prevent formation of air bubbles within the channels. By controlling the loading of the channel of each layer, the loss of cells during the loading process can be prevented. Also, by preventing the formation of bubbles within the channels, the fluidic resistance between channels would not be significantly altered, and cells may be exposed to vectors in a more homogenous manner. Thus, the devices according to the disclosure can be configured to load each layer sequentially to avoid bubbles to preserve homogeneity of vector exposure, mitigate cell loss, while using less vector and shorter transduction times by virtue of enabling more efficient interactions of cells and vector.

As used herein, the testing fluid may include cells and vector. In some embodiments, the cells may be of any type. For example, the cells may include but are not limited to human T cells, primary human cells, immortalized cell lines, CD34+ hematopoietic stem cells, induced pluripotent stem cells (iPSCs), non-adherent/suspension cells, adherent cells, among others, or any combination thereof.

In some embodiments, the vector may include a carrier, nucleic acids, among others, or any combination thereof. For example, the vector may include but are not limited to non-viral vectors, viral vectors, among others, and any combination thereof. For example, non-viral vectors may include but are not limited to liposomes, spheroplasts, red blood cell ghosts, colloidal metals, calcium phosphate, DEAE Dextran plasmids, among others, or a combination thereof. The viral vectors may include but are not limited to retroviral vectors, lentiviral vectors, pseudotype vectors, adenoviral vectors, adeno-associated viral vectors, among others, and any combination thereof.

In some embodiments, the systems, devices, and methods can be configured for fluid-based transduction protocols. For example, the protocols may include flow transduction (i.e., continuous perfusion), static (e.g., single loading), among others, or a combination thereof.

As used herein, the term “communicate” or “connect” (e.g., a first component “communicates with” or “is in communication with” a second component) or “fluid communication” and grammatical variations thereof are used herein to indicate a fluidic relationship between two or more components and/or channel segments. As such, the fact that one component/channel segment is said to communicate with a second component/channel segment is not intended to exclude the possibility that additional components may be present between, and/or operatively associated or engaged with, the first and second components.

In some embodiments, the device may include more than one layer. The layers may form a stack of layers so that each layer within the stack of layers substantially covers the preceding layer in the stack.

In some embodiments, the more than one of the layers may include a fluid path (e.g., also referred to as “fluid layer”). For example, the device may include a first layer having a fluid path and a second layer having a fluid path disposed on a solid (bottom) substrate. In some embodiments, the device may include any number of (fluid) layers, such as four layers, five layers, more than five layers (e.g., 10, 20, 30, etc.), among others, or a combination thereof.

In some embodiments, each of the first layer and second layer may include an entry region configured to receive a testing fluid (e.g., cell/vector mixture) loaded into the device, an exit region for collecting the transduced cells, and a channel disposed at least between the entry region and the exit region. In some embodiments, the channel may extend beyond the entry region and the exit region. The channel may correspond to a (horizontal) fluid passageway or fluid path defined in the layer. The layers may be stacked and disposed (or connected) in parallel so that each channel/fluid passageway/fluid path is horizontal.

In some embodiments, the device may include at least one loading region (e.g., inlet) for loading the fluid into the one or more channels of the device and at least one collection region (e.g., outlet) for collecting the cells from the device. In some embodiments, the device can include: at least one layer that includes at least one entry region of at least one layer that acts as the at least one loading region; at least one layer that includes at least one loading region and an entry region; at least one layer having another configuration; or any combination thereof.

In some examples, the top layer of the device may include the at least one loading region that communicates with each entry region of the remaining (fluid) layers disposed below the top layer. In this example, the layers may be stacked and disposed (or connected) in parallel such that the center of each entry region may be aligned and connected with the center of the at least one loading region. This way, the loading region and the entry regions may form a vertical path or passageway for loading the cells/viral vectors to each channel/layer.

In further examples, additional layer(s) of the device may additionally include the at least one loading region and the entry region. For example, the layers may be stacked and disposed (or connected) in parallel such that the center of the loading regions may be aligned and connected to form a vertical path or passageway and each entry region may be aligned to form a vertical path or passageway. In this example, the device may be configured to be “bottom” loaded (e.g., loading the bottom (fluid) channel first before the (fluid) channels disposed above).

In some embodiments, the device may include: at least one layer in which at least one exit region of at least one layer acts as the at least one collection region; at least one layer of that includes at least one collection region and an exit region; at least one layer having another configuration; or any combination thereof.

In some embodiments, the top layer of the device may include the collection region that communicates with each of the exit regions of the remaining (fluid) layers disposed below the top layer. For example, the center of each exit region may be aligned and connected with the center of the collection region (of the top layer). This way, the collection region and the exit regions form a vertical fluid path/passageway for retrieving the cells/viral vectors from each channel/layer.

In further examples, the top layer may also include an exit region and a channel disposed between the exit region and the collection region. In this example, the top layer may act as an overflow layer. In this example, the center of each exit region may be aligned and connected to form a vertical fluid path/passageway to the overflow layer for collecting the cells/viral vectors to be retrieved via the collection region. This way, when loading with a testing fluid (e.g., cell/virus mixture), the overflow layer may prevent losing cells if the device is overloaded (e.g., fluid is pushed too far through the channels) while still enhancing transduction by virtue of minimizing diffusion distance of viral vectors to cells.

Each loading region, collection region, entry region, exit region, and channel may have one or more dimensions. For example, the dimensions of each loading region, collection region, entry region, and/or exit region may include a width or diameter, a height, among others, or a combination thereof. In some embodiments, each channel of the device may have channel dimensions. The channel dimensions may include height, width, and a length.

In some embodiments, the channel dimensions, such as width and/or length, and/or region dimensions, such as width or diameter, may differ between the layers so that the device may include a taper of these dimensions between horizontal fluid paths and/or along the vertical fluid path. For example, a taper of dimensions between the horizontal fluid paths may include the channel width and/or length differing between layers and the channel height of all channels of the device may be substantially the same. By way of another example, for a taper of the dimensions along the vertical fluid path, the dimension(s) (e.g., width or diameter) of the entry region and/or the exit region may differ between one or more layers.

In some embodiments, the height of the channels of a device may be within a range from about 50-200 μm. In some embodiments, the length of the channels of a device may be within a range of about 1 cm to about 20 cm or more than 20 cm. In some embodiments, the width of the channels of device may be within a range of about 1 mm to 5 mm or more than 5 mm. The surface area of each channel may range from about 0.3 cm² to 100 cm² or more than 100 cm². The volume of each channel may range from about 5 μL to 2 mL or more than 2 mL.

In some embodiments, for each layer, the diameter of the exit region may be different so that the corresponding vertical path tapers along its length. By way of example, the diameter/width of the exit region may decrease from the layer closest to the collection region (i.e., top layer) to the layer farthest from the collection region (i.e., the bottom layer). In this example, the layer closest to the top layer (i.e., closest to the collection region) has an exit region having a diameter/width larger than the exit region of the layer closest to the bottom layer (i.e., furthest from the top layer/collection region).

In some embodiments, for each layer, the diameter of the entry region may alternatively or additionally be different so that the corresponding vertical path alternatively or additionally tapers along its length. For example, the diameter/width of the entry region may increase from the layer farthest from the loading region (e.g., i.e., the bottom layer) to the layer closest to the collection region (i.e., the top layer). In this example, the layer closest to the top layer (i.e., closest to the collection region) has a loading region having a diameter/width smaller than the entry region of the layer closest to the bottom layer (i.e., furthest from the top layer/collection region).

In another example, the layer closest to the inlet and outlet (i.e., top layer) may have a loading region with the smallest diameter and an exit region with the largest diameter; and the layer furthest from the layer including the collection region (i.e., the layer closest to the bottom layer) may have a loading region with the largest diameter and an exit region with the smallest diameter. In this example, the device may include two vertical paths having a taper in region diameters.

By way of another example, one or more of the channel dimensions may be additionally or alternatively different between one or more (fluid) layers so that the surface area of the channel of each layer tapers with respect to the layers. For example, the surface area of the channel closest to the bottom layer (e.g., furthest from the collection region) may be smaller than the surface area of the channel closest to the layer having the collection region (e.g., top layer). The difference between the surface area of the channel of each layer may be based on a difference between one or more channel dimensions of the respective layer. For example, the difference in channel surface area between layers may be based on the aspect ratio determined from the different channel length and/or width. The height between layers may be the same. For example, the channels may have the same height, same and/or different length and/or width, same shape/pattern, other dimensions, among others, or a combination thereof. The channel may have any shape/pattern.

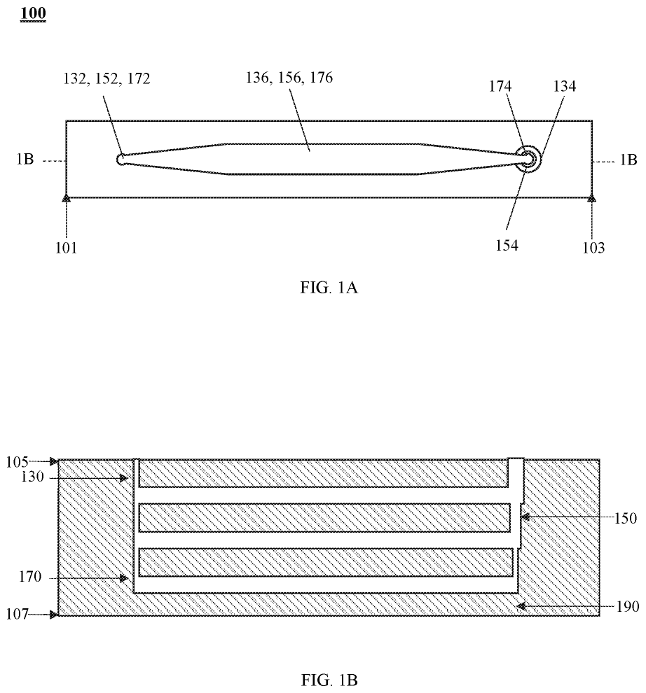

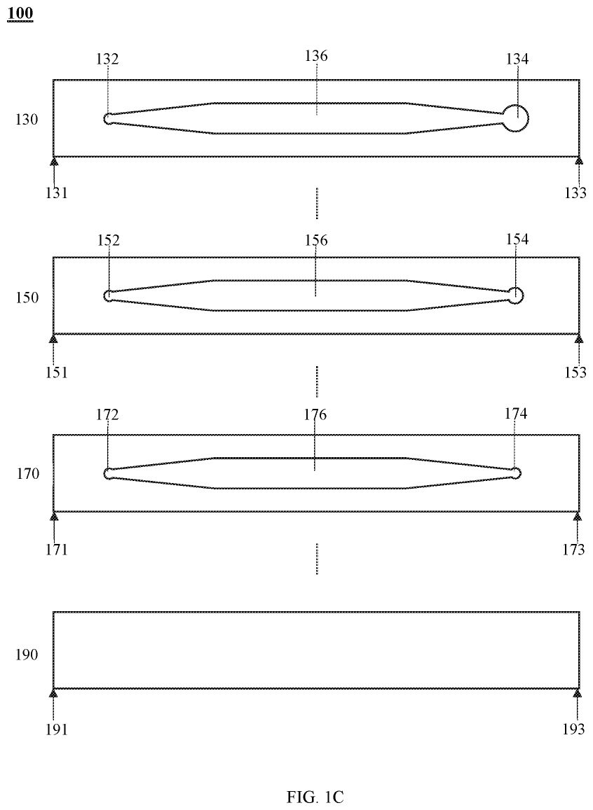

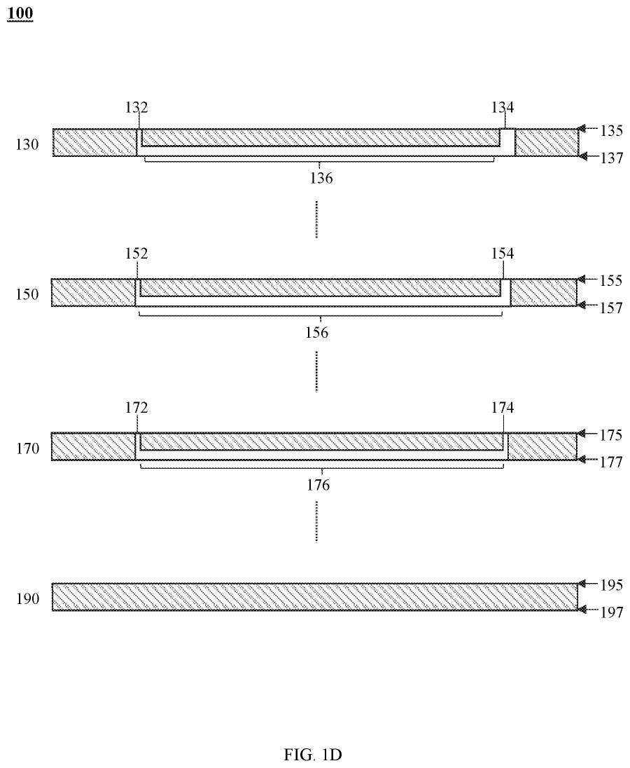

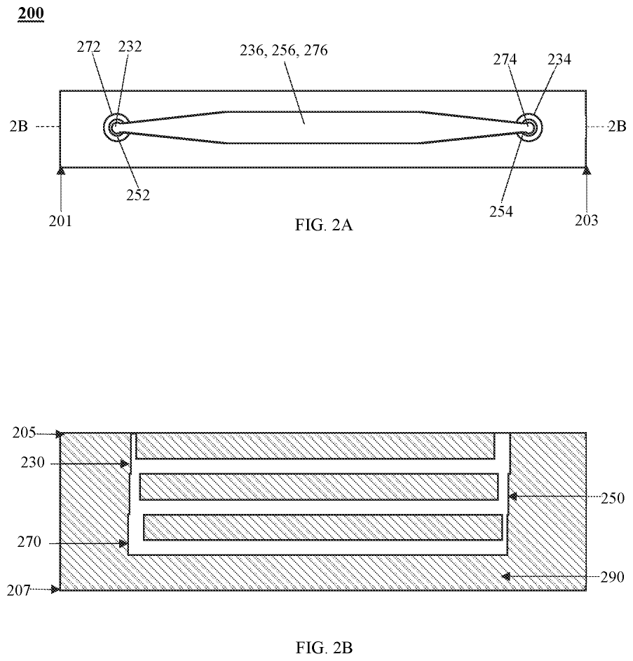

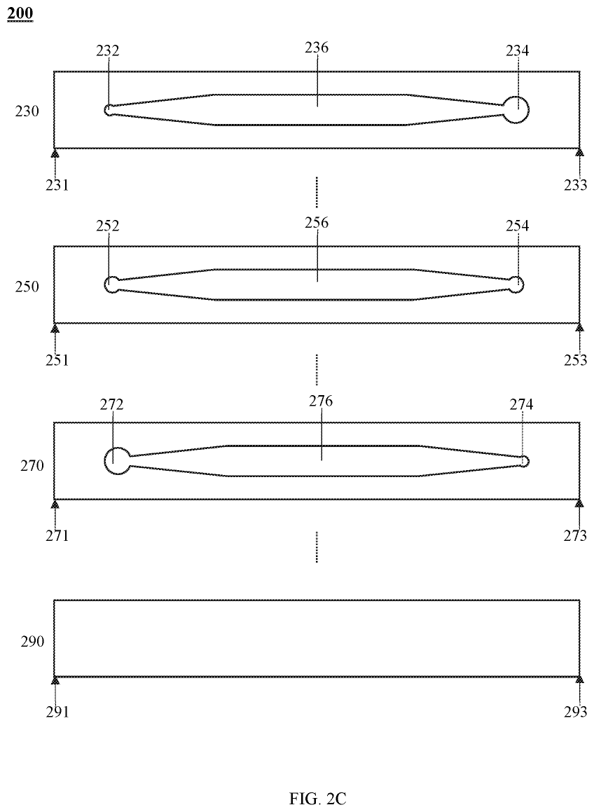

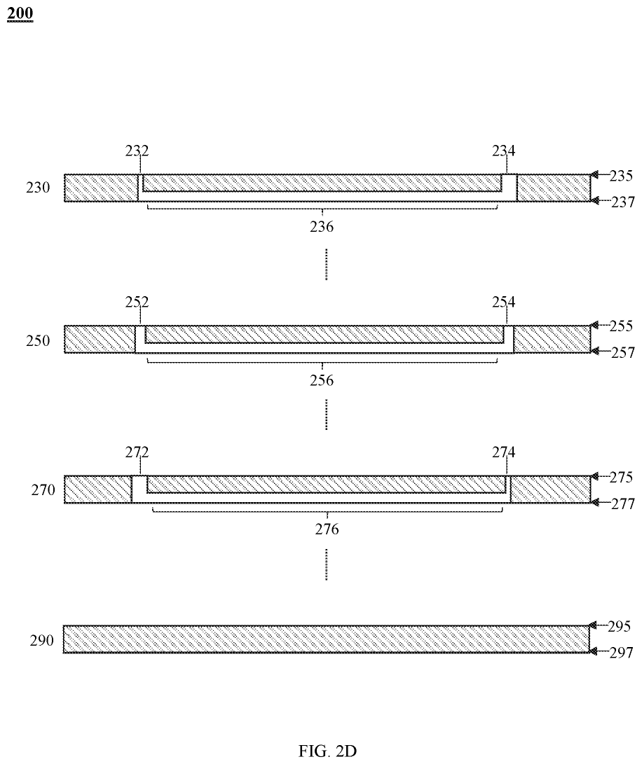

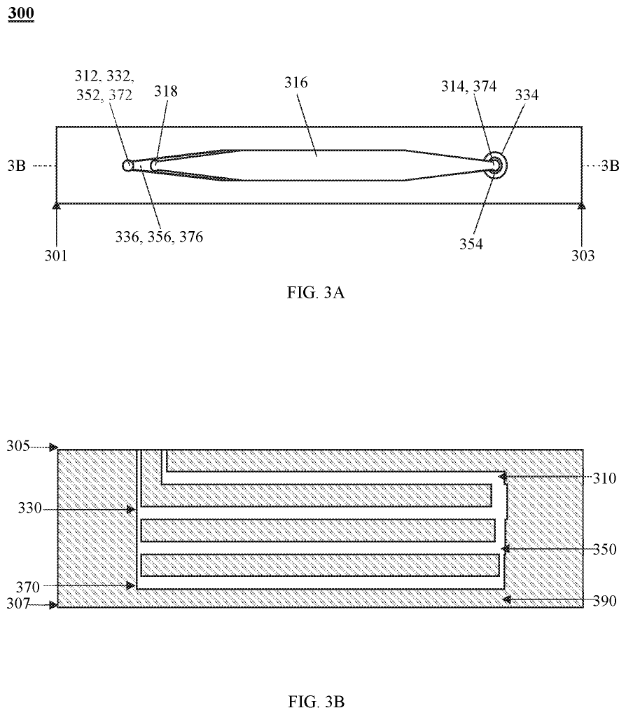

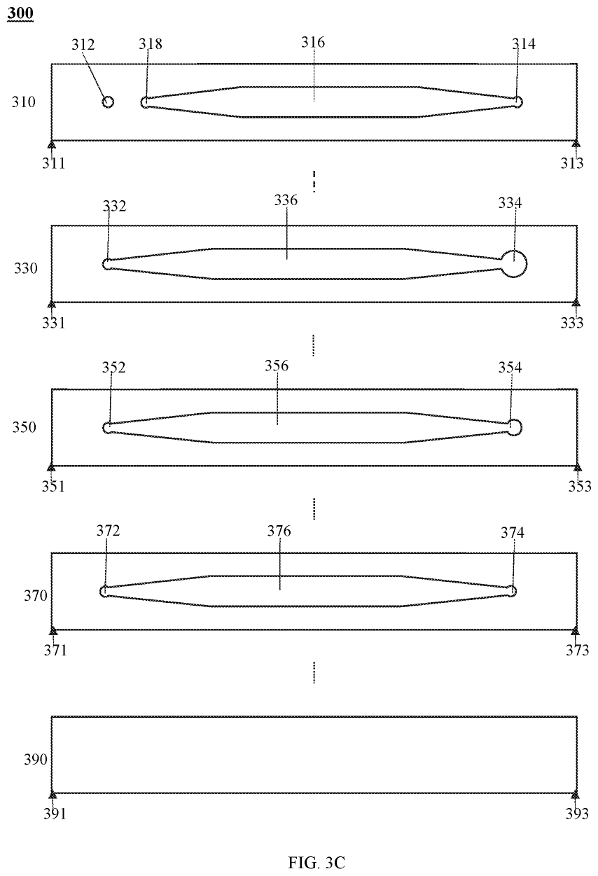

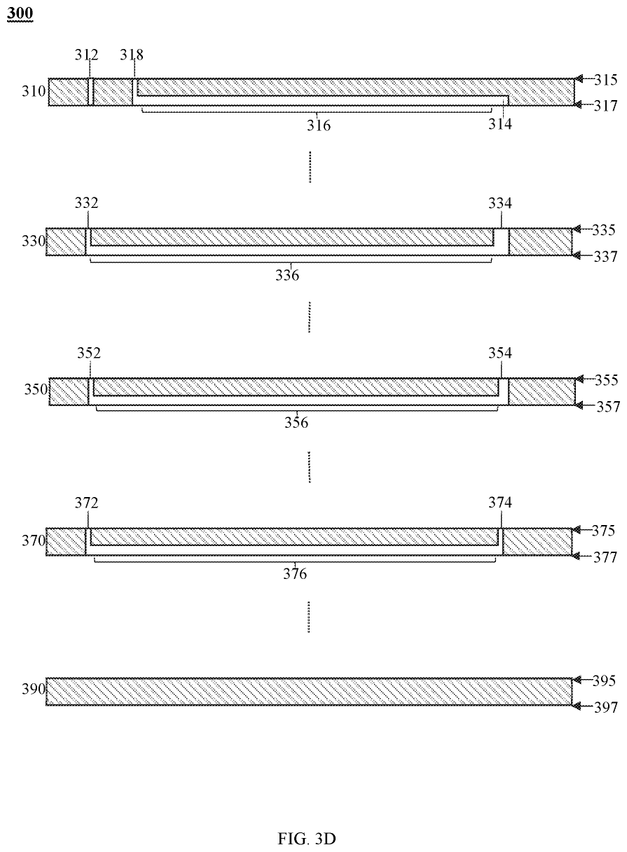

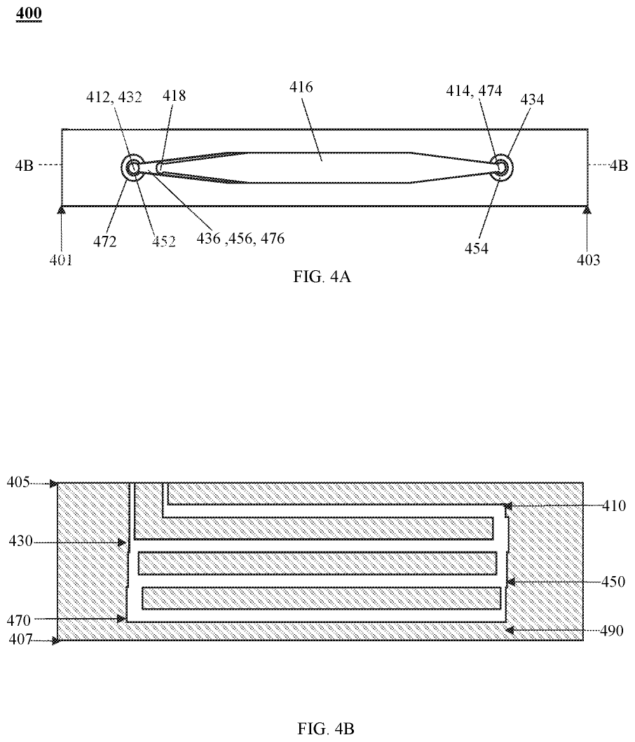

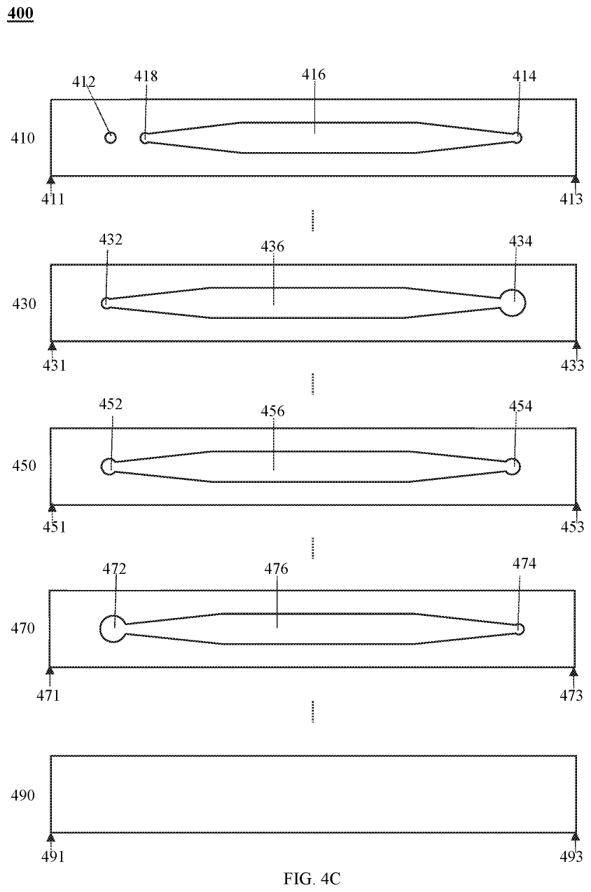

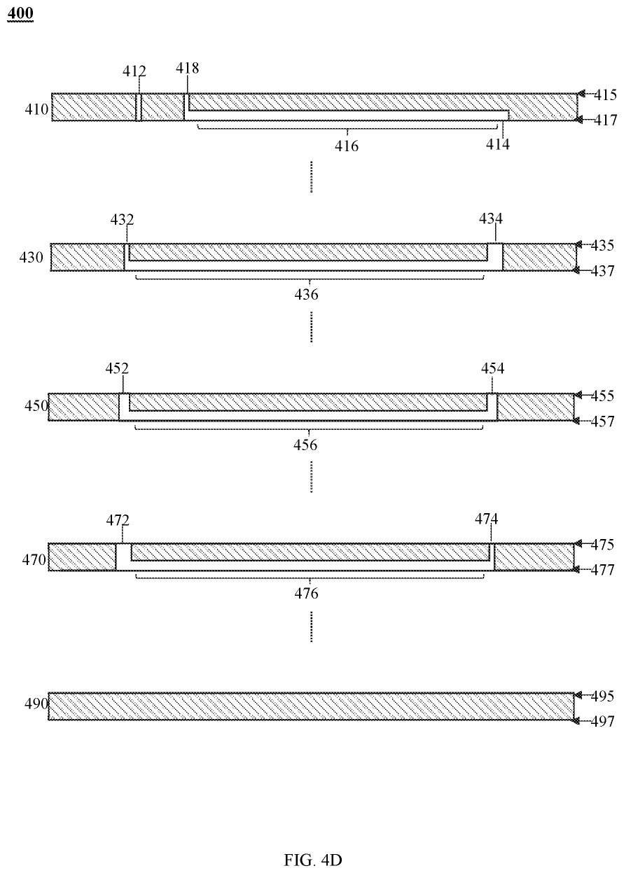

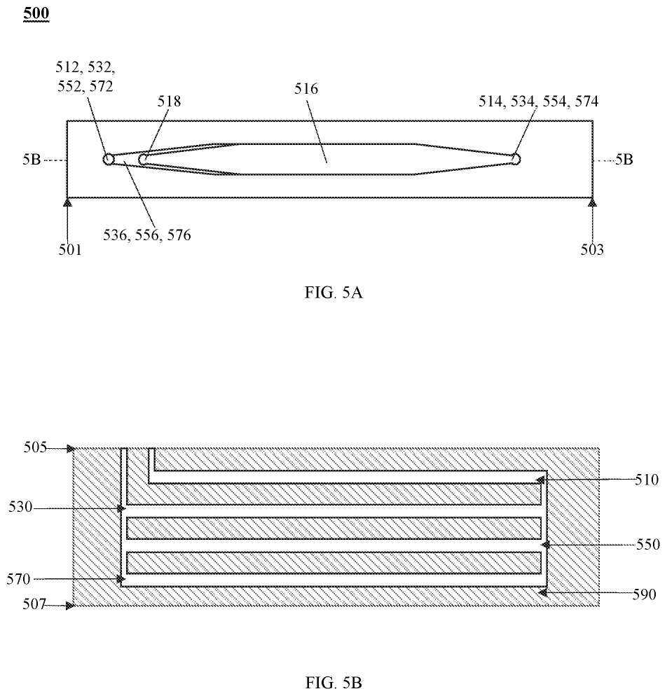

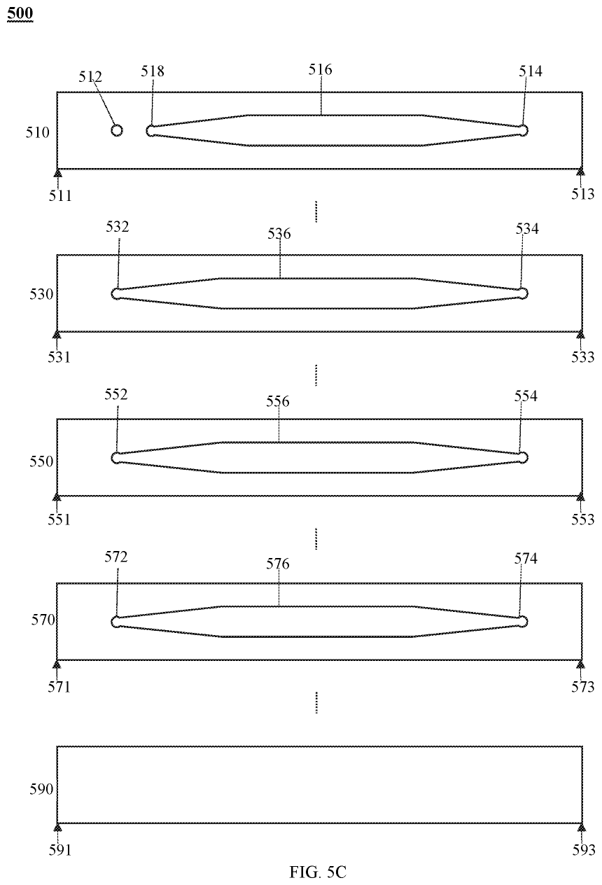

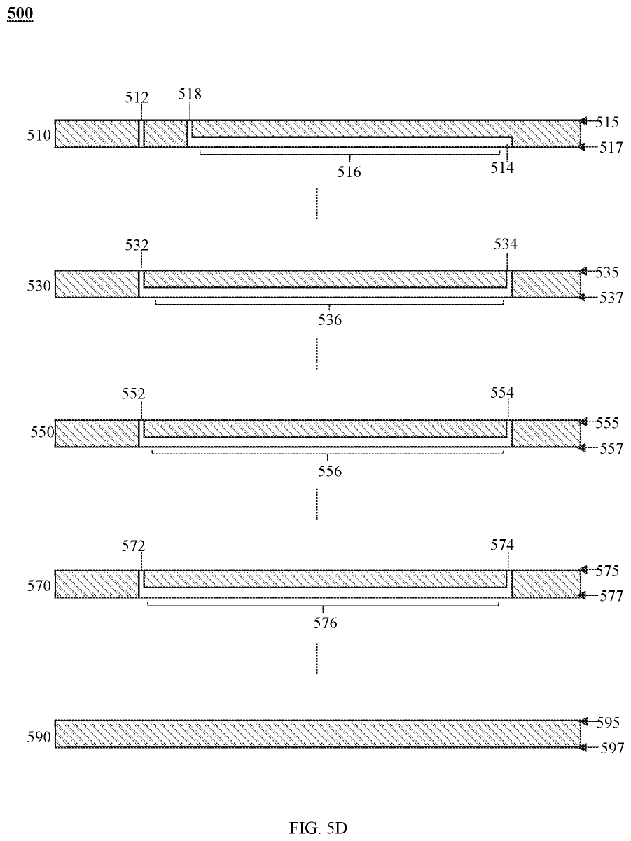

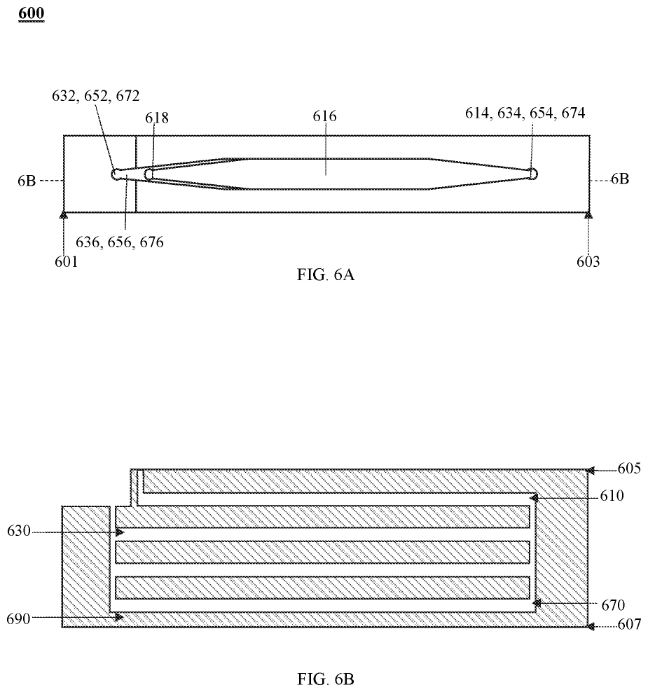

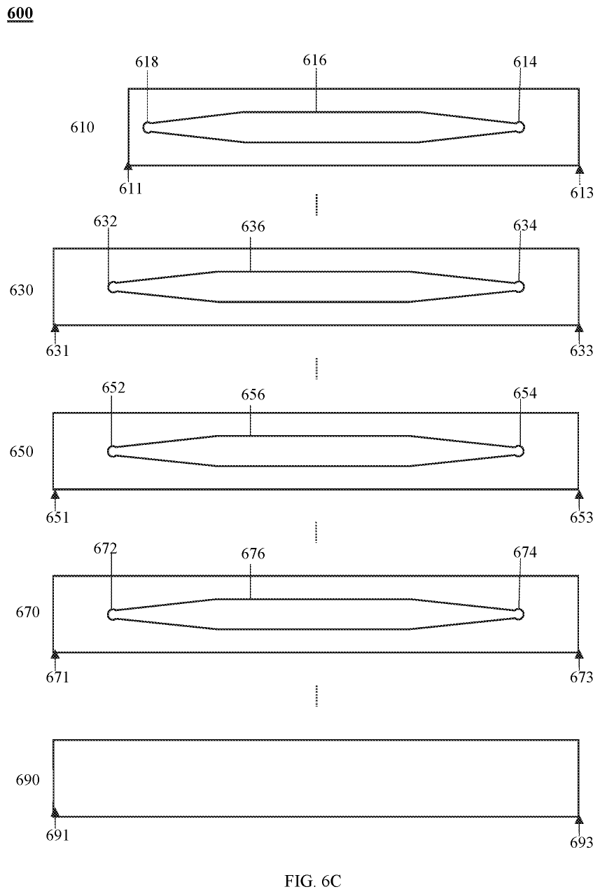

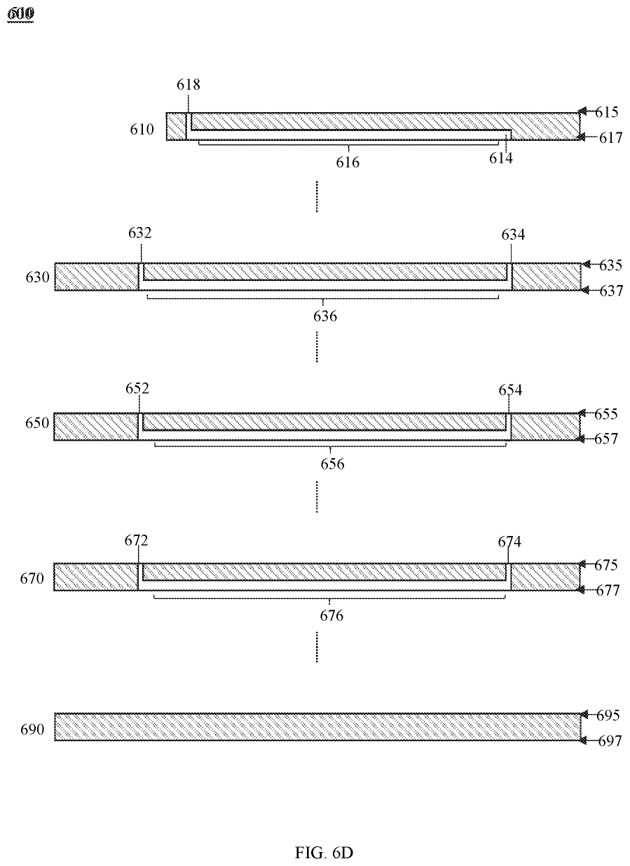

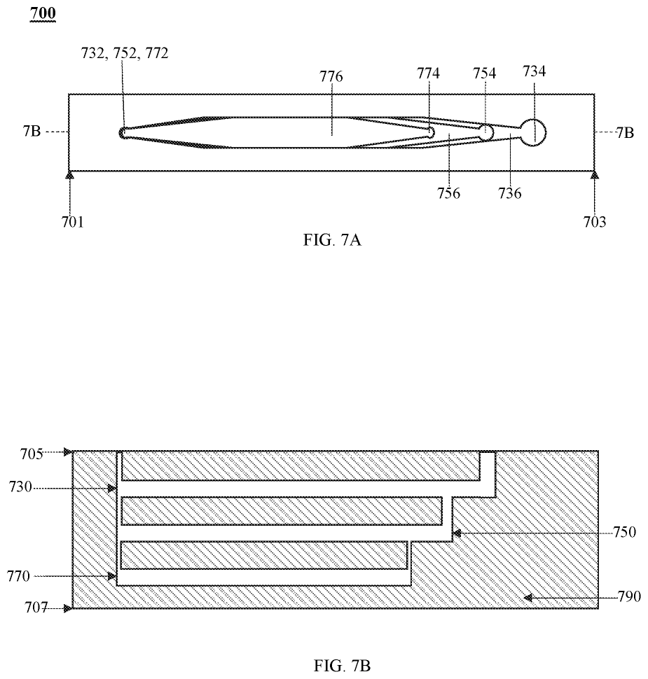

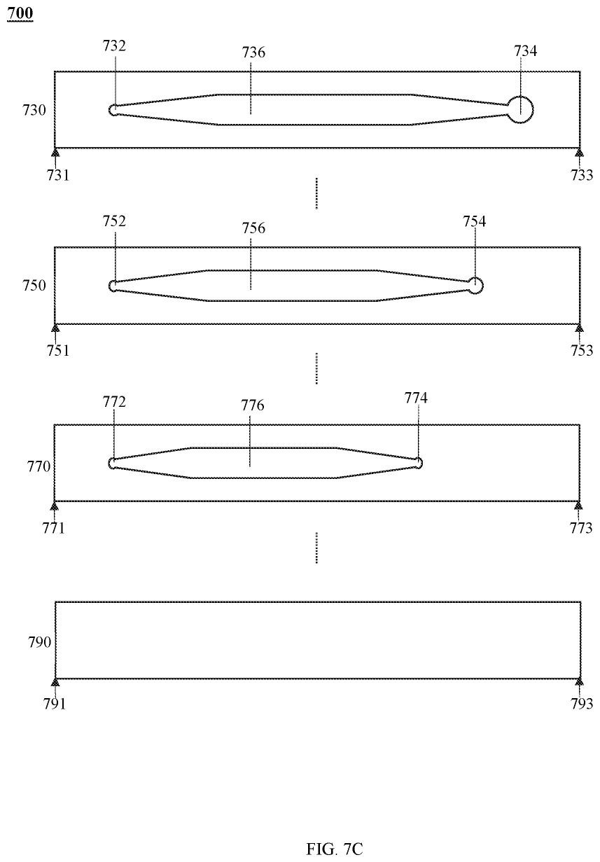

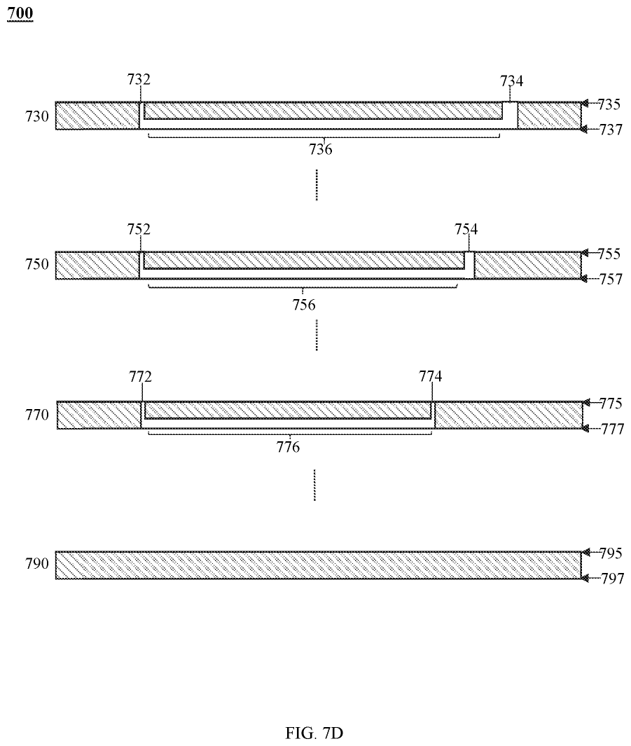

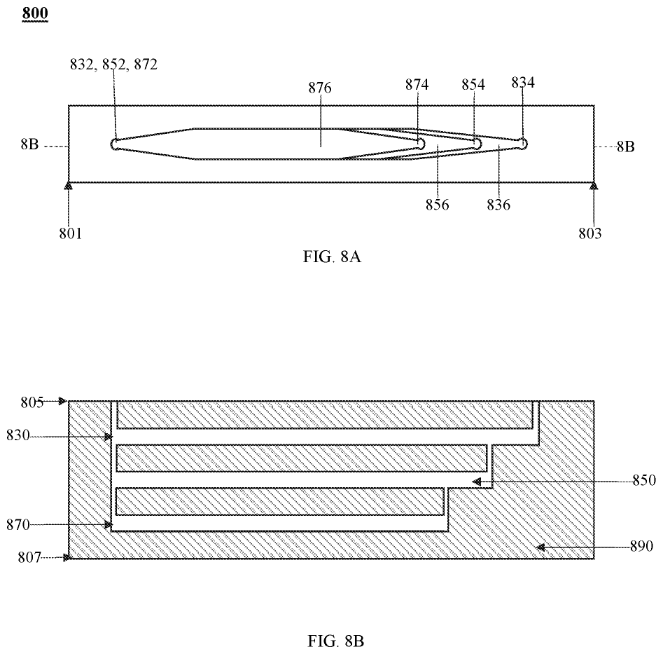

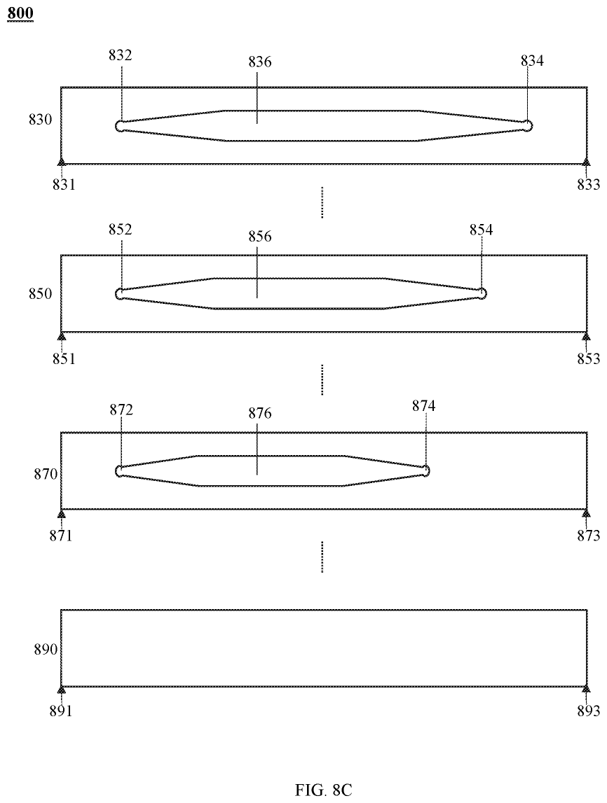

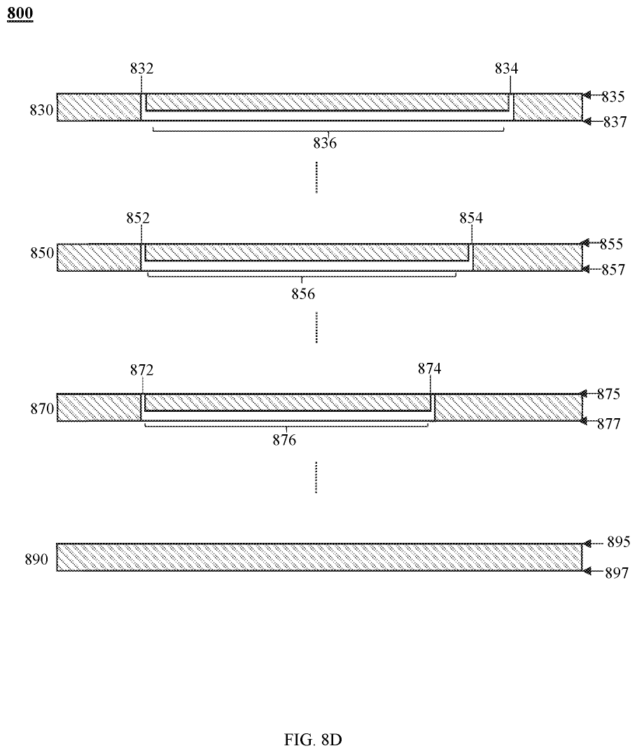

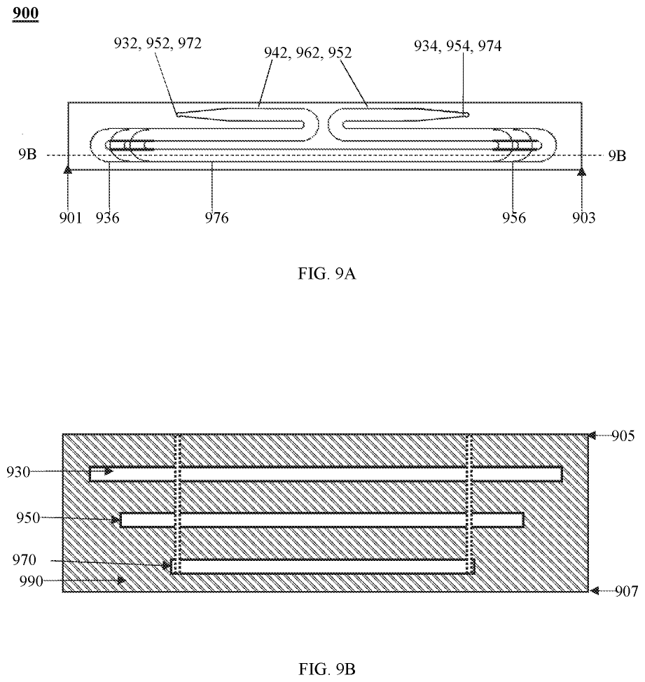

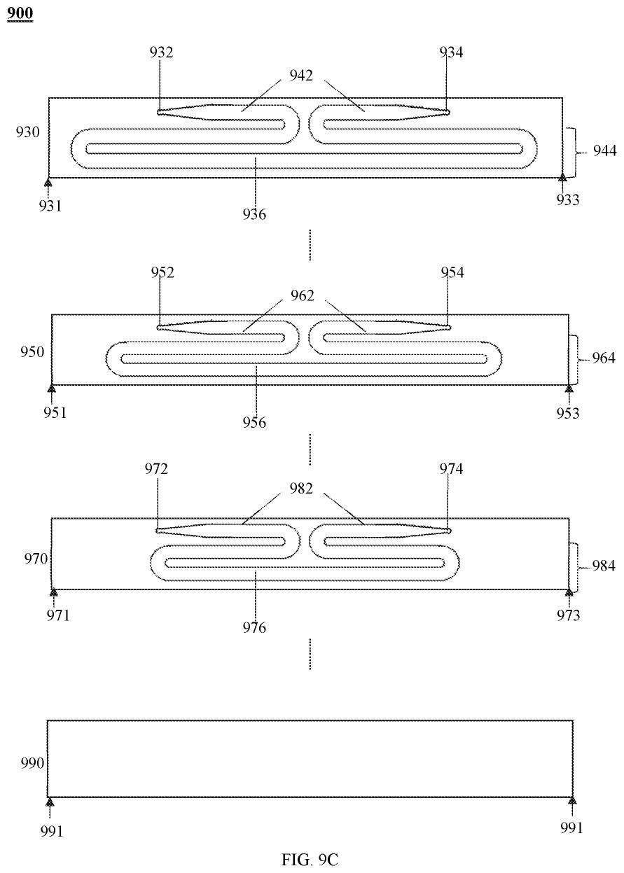

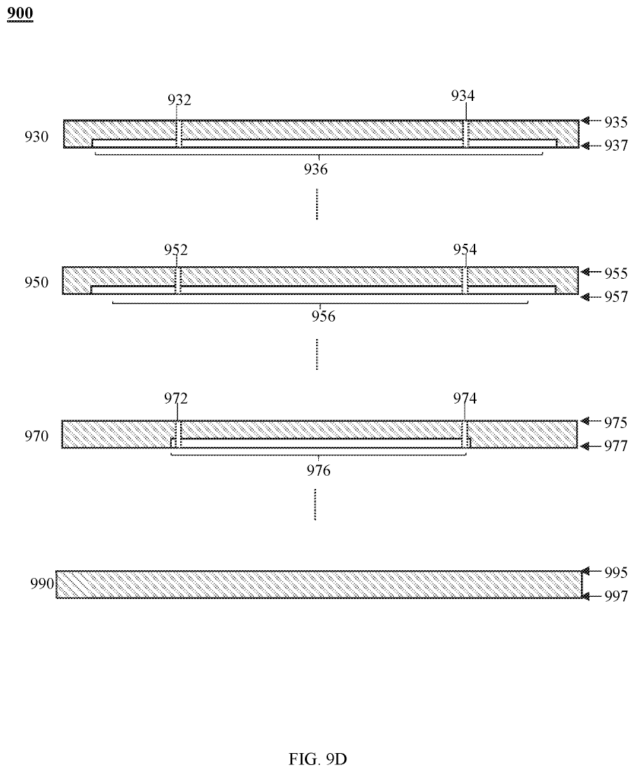

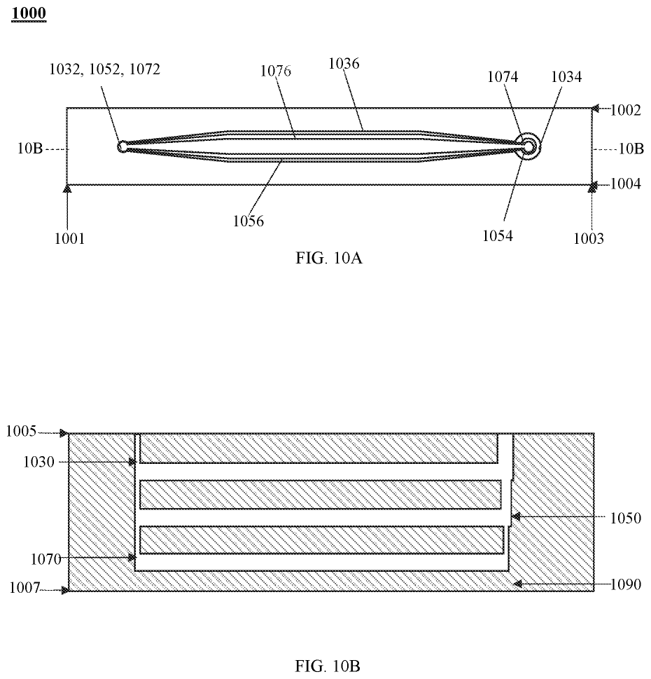

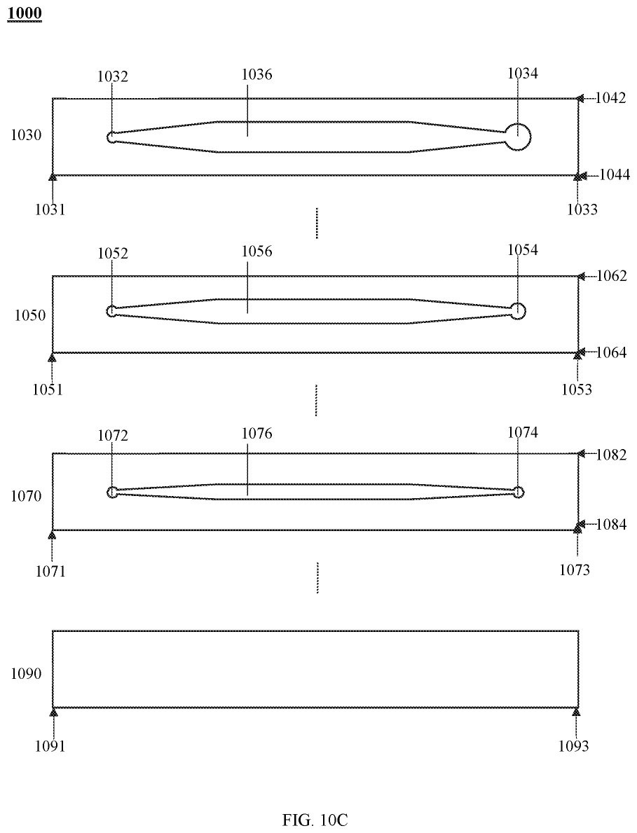

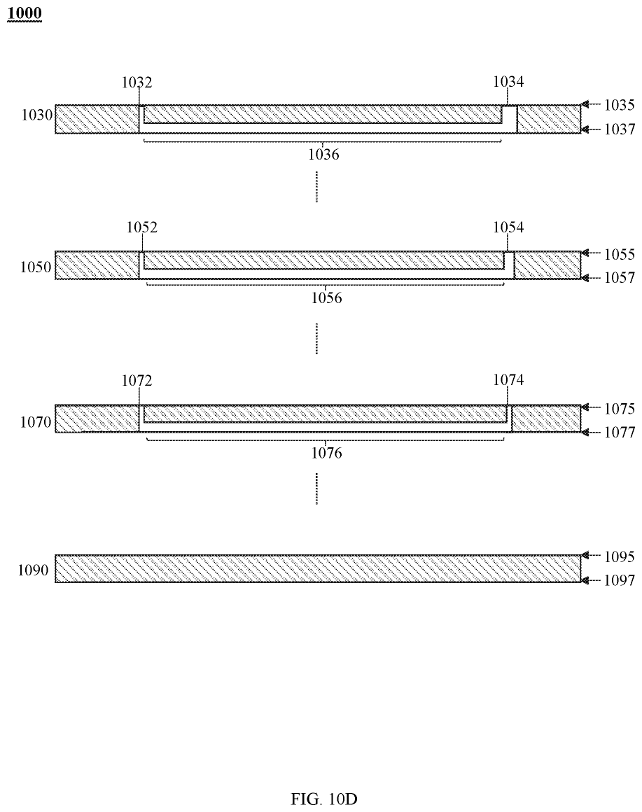

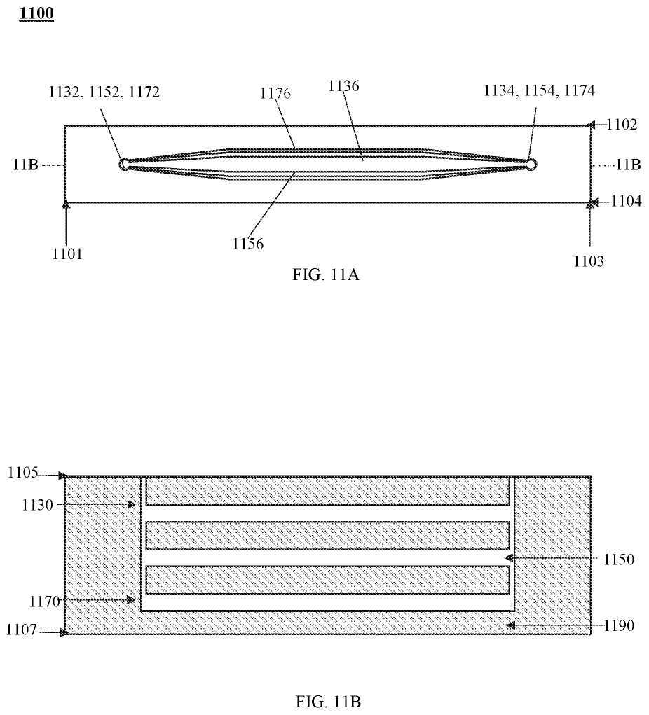

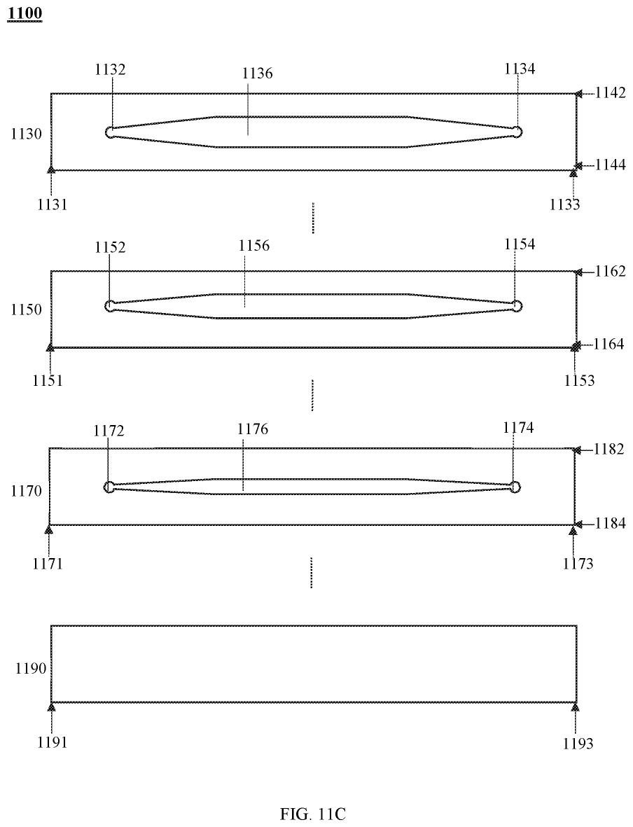

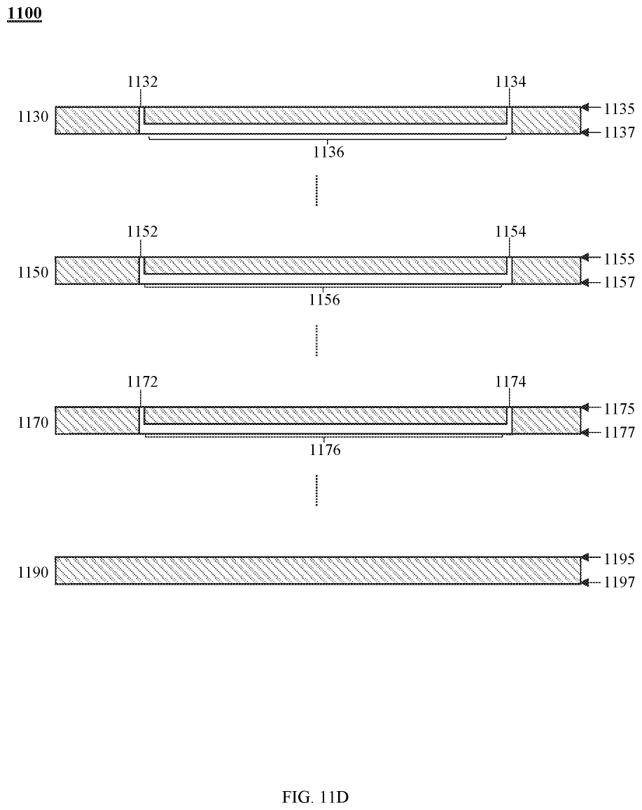

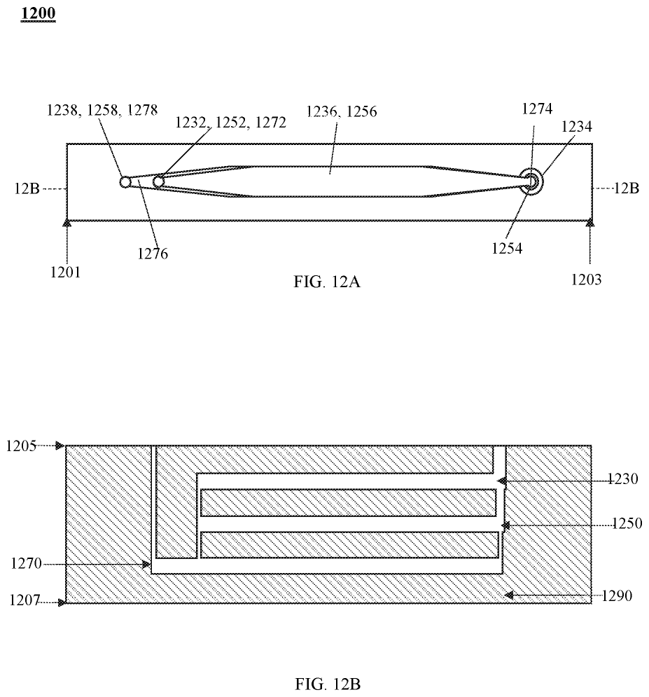

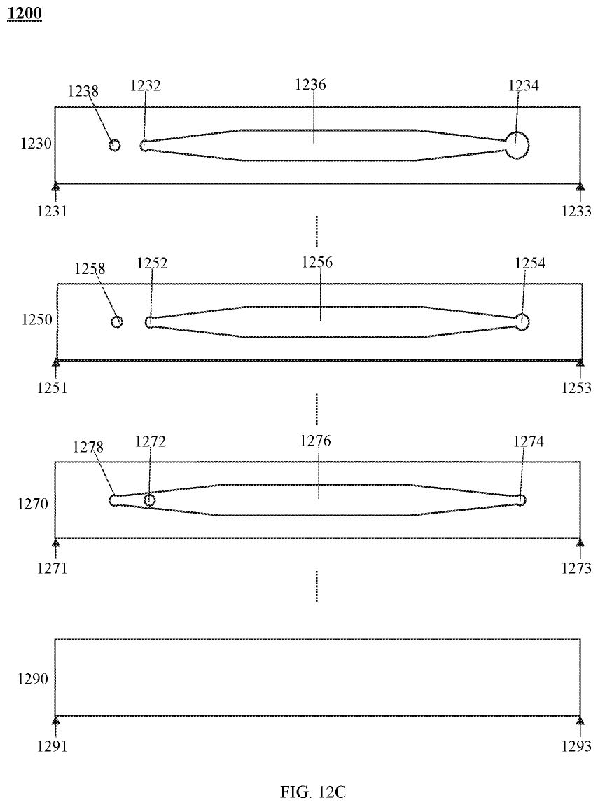

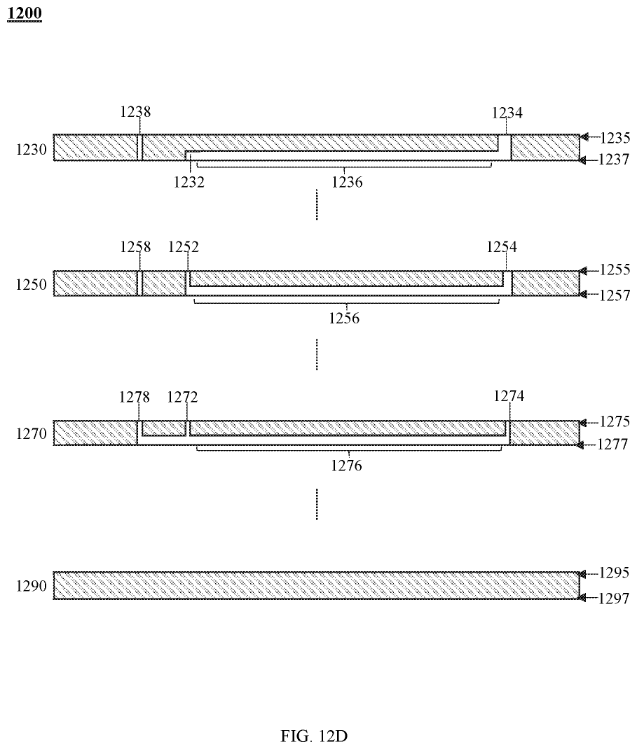

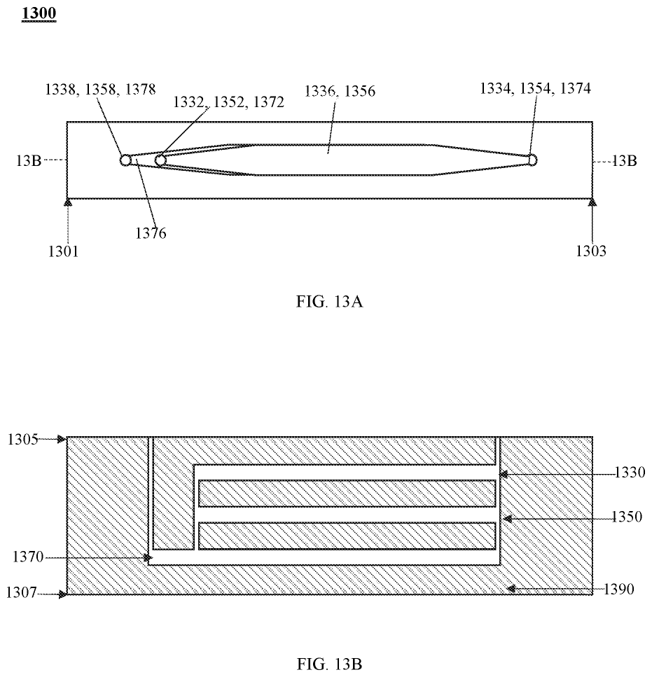

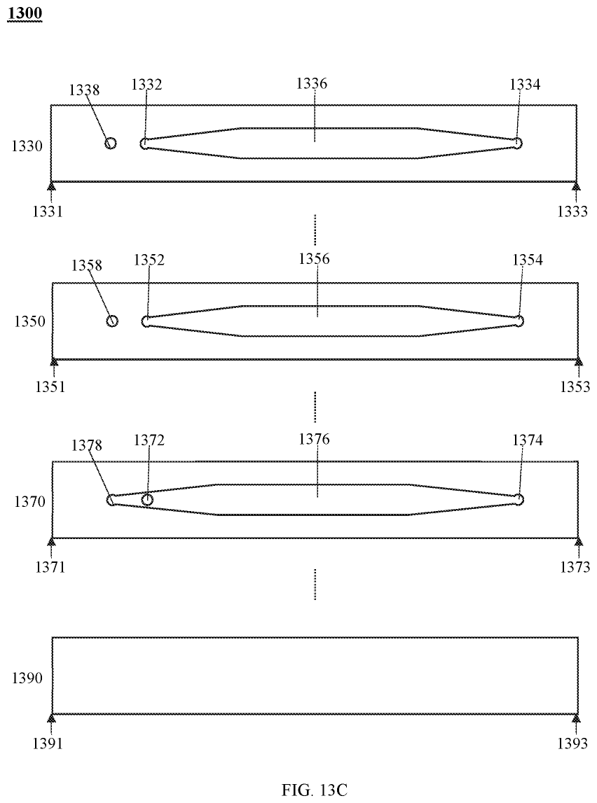

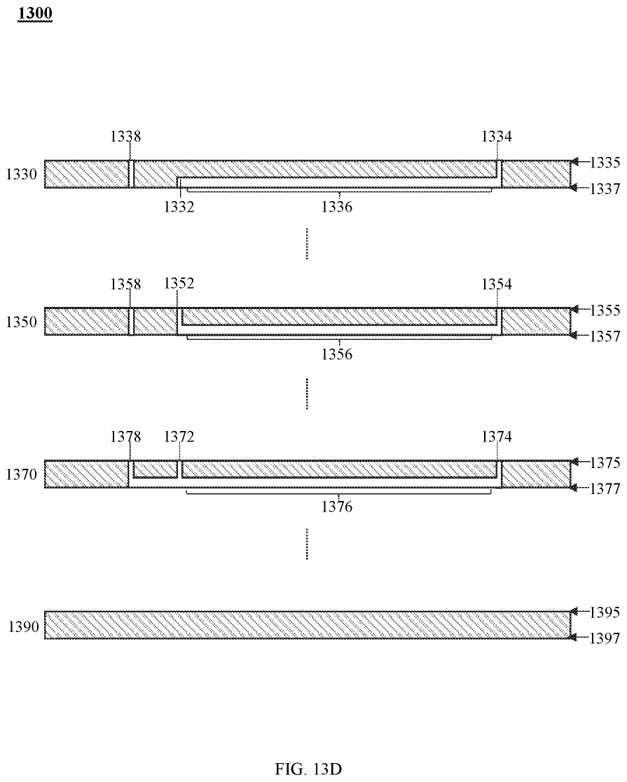

FIGS. 1A-13D show examples of devices according to some embodiments. FIGS. 1A-2D show examples of devices having tapered vertical paths; FIGS. 3A-4D show examples of devices having an overflow layer and tapered vertical paths; FIGS. 5A-5D show an example of a device having an overflow layer; FIGS. 6A-D show another example of a device having an overflow layer; FIGS. 7A-7D show an example of a device having tapered vertical and horizontal paths; FIGS. 8A-9D show examples of devices having tapered horizontal path; FIGS. 10A-D show another example of a device having tapered vertical and horizontal paths; FIG. 11A-11D show an example of a device having tapered horizontal path; FIGS. 12A-12D show an example of a device being configured for bottom-loading the horizontal paths and having a tapered vertical path; and FIGS. 13A-D show another example of a device being configured for bottom-loading the horizontal paths. It will be understood that a device according to embodiments are not limited to the configuration and/or combination of vertical paths, horizontal paths, other features (e.g., overflow layer and/or bottom-loaded configuration) as shown and described with respect to the features. The devices according to embodiments may include any one feature or any combination of features of the vertical paths, horizontal features, channel shape and/or pattern (e.g., looped vs. elongated shape described, as well as additional and/or alternative features.

The devices shown and described in the FIGS. 1A-13D may also include more or less (fluid) layers than shown in the examples. The devices may include any number of stacked layers, such as three layers, four layers, five layers, six layers, more than six layers (e.g., ten layers), etc.

The number of layers and/or the features of the devices discussed therein can be tailored to suit the numbers of cells to be processed. This way, the devices according to embodiments can be scalable to provide greater flexibility and greater potential cell capacity.

FIGS. 1A-D show views of a device 100 having a tapered vertical path along the exit regions of the layers according to embodiments. As shown in the top view shown in FIG. 1A, the device 100 may have a first end 101, a second end 103, and a length therebetween. As shown in FIG. 1B, the device 100 may have a first side 105, a second side 107, and a height therebetween. As shown in FIG. 1B, the device 100 may include a plurality of stacked layers disposed vertically in parallel. In this example, the device 100 may include (top) (fluid) layer 130, a (fluid) layer 150, and a (fluid) layer 170 disposed above a (bottom) layer 190.

In this example, each fluid layer may include a fluid path disposed horizontally with respect to the length of the device. Each fluid layer may include an entry region, an exit region, and a channel that extends therebetween disposed horizontally with respect to the device length.

As shown in exploded top view in FIG. 1C, the layer 130 may include a first end 131, a second end 133, and a length therebetween. The layer 130 may include a fluid path. The fluid path of the layer 130 may include an entry region 132, an exit region 134, and a channel 136 disposed there between along the length of the layer 130. The layer 130 may have a first side 135, a second side 137, and a height therebetween as shown in the cross-sectional exploded view in FIG. 1D.

Like the layer 130, the layer 150 may include a first end 151, a second end 153, and a length therebetween as shown in FIG. 1C. The layer 150 may include a fluid path. The fluid path of the layer 150 may include an entry region 152, an exit region 154, and a channel 156 disposed there between along the length of the layer 150. The layer 150 may have a first side 155, a second side 157, and a height therebetween as shown in the cross-sectional exploded view in FIG. 1D.

Like the layers 130 and 150, the layer 170 may include a first end 171, a second end 173, and a length therebetween. The layer 170 may include a fluid path. The fluid path of the layer 170 may include an entry region 172, an exit region 174, and a channel 176 disposed there between along the length of the layer 170. The layer 170 may have a first side 175, a second side 177, and a height therebetween as shown in the cross-sectional exploded view in FIG. 1D.

The bottom layer 190 may include a first end 191, a second end 193, and a length therebetween; and a first side 195, a second side 197, and a height therebetween as shown in the cross-sectional exploded view in FIG. 1D.

In this example, the entry region 132 of the layer 130 may correspond to the loading region and the exit region 134 of the layer 130 may correspond to the collection region. As shown in FIG. 1B, the layers 130, 150, 170, and 190 may be stacked so that the centers of the entry regions 152 and 172 are aligned with and connected to the center of the entry region/loading region 132 and the centers of the exit regions 154 and 174 are aligned with and connected to the center of the exit region/collection region 134. The layers 130, 150, and 170 may have channels 136, 156, 176 with the same channel dimensions (height, width, and length) and entry regions 132, 152, and 176 with the same dimensions (e.g., height and width/diameter).

In this example, the layers 130, 150, and 170 may differ with respect to the width/diameter of the exit regions 134, 154, and 174. As shown in FIGS. 1A-1D, the width/diameter of the exit region 134 may be larger than the width/diameter of the exit region 154, and the width/diameter of the exit region 154 may be larger than the width/diameter of the exit region 174. This way, the width/diameter of the exit region tapers from larger to smaller along with the vertical path defined by the exit regions 134, 154, and 174 from the first side 105 to the second side 107.

In use, when loading a testing fluid into the device 100 via the region 132, the vertical path (entry regions 132, 152, 172) may be filled first before the respective horizontal path (e.g., channels 136, 156, 176) due to the lower resistance. Once the vertical path defined by the entry regions has been filled, the respective horizontal paths (e.g., channels) may then begin to fill. In use, the channels could fill at substantially the same rate, with a slight bias to fill the bottommost channel (176) first and the topmost channel (136) last due to the slight differences in resistance. If the channels fill at slightly different rates out of order, any overflow can be collected in the vertical path defined by the exit regions (134, 154, 174). Due to the tapered exit regions, any air bubbles that form from differential channel filling can dissipate. The decreasing exit region diameter prevents air bubbles from getting trapped.

FIGS. 2A-D show views of a device 200 having a tapered vertical path along the entry regions and the exit regions of the layers according to embodiments. As shown in the top view shown in FIG. 2A, the device 200 may have a first end 201, a second end 203, and a length therebetween. As shown in FIG. 2B, the device 200 may have a first side 205, a second side 207, and a height therebetween. As shown in FIG. 2B, the device 200 may include a plurality of stacked layers disposed vertically in parallel. In this example, the device 200 may include (top) (fluid) layer 230, a (fluid) layer 250, and a (fluid) layer 270 disposed above a (bottom) layer 290.

As shown in exploded top view in FIG. 2C, the layer 230 may include a first end 231, a second end 233, and a length therebetween. The layer 230 may include a fluid path. The fluid path of the layer 230 may include an entry region 232, an exit region 234, and a channel 236 disposed there between along the length of the layer 230. The layer 230 may have a first side 235, a second side 237, and a height therebetween as shown in the cross-sectional exploded view in FIG. 2D.

Like the layer 230, the layer 250 may include a first end 251, a second end 253, and a length therebetween as shown in FIG. 2C. The layer 250 may include a fluid path. The fluid path of the layer 250 may include an entry region 252, an exit region 254, and a channel 256 disposed there between along the length of the layer 250. The layer 250 may have a first side 255, a second side 257, and a height therebetween as shown in the cross-sectional exploded view in FIG. 2D.

Like the layers 230 and 250, the layer 270 may include a first end 271, a second end 273, and a length therebetween. The layer 270 may include a fluid path. The fluid path of the layer 270 may include an entry region 272, an exit region 274, and a channel 276 disposed there between along the length of the layer 270. The layer 270 may have a first side 275, a second side 277, and a height therebetween as shown in the cross-sectional exploded view in FIG. 2D.

The bottom layer 290 may include a first end 291, a second end 293, and a length therebetween; and the layer 290 may have a first side 295, a second side 297, and a height therebetween as shown in the cross-sectional exploded view in FIG. 2D.

In this example, the entry region 232 of the layer 230 can correspond to the loading region and the exit region 234 of the layer 230 can correspond to the collection region. As shown in FIG. 2B, the layers 230, 250, 270, and 290 may be stacked so that the centers of the entry regions 252 and 272 are aligned with and connected to the center of the entry region/loading region 232 and the centers of the exit regions 254 and 274 are aligned with and connected to the center of the exit region/collection region 234. The channels 236, 256, and 276 of the layers 230, 250, and 270, respectively may have the same channel dimensions (height, width, and length).

In this example, the layers 230, 250, and 270 may differ with respect to the width/diameter of the entry regions 232, 252, and 272 and the width/diameter of the exit regions 234, 254, and 274. As shown in FIGS. 2A-2D, the width/diameter of the entry region 232 may be smaller than the width/diameter of the entry region 252, and the width/diameter of the entry region 252 may be smaller than the width/diameter of the entry region 272. Like the device 100, the width/diameter of the exit region 234 may be larger than the width/diameter of the exit region 254, and the width/diameter of the exit region 254 may be larger than the width/diameter of the exit region 274. This way, the width/diameter of the entry regions can taper from smaller to larger along the vertical path defined by the entry regions 232, 252, and 272 from the first side 205 to the second side 207 and the width/diameter of the exit region can taper from larger to smaller along the vertical path defined by the exit regions 234, 254, and 274 from the first side 205 to the second side 207.

In use, like the device 100, when loading a testing fluid into the device 200 via the region 232, the vertical path (entry regions 232, 252, 272) may be filled first before the respective horizontal path (e.g., channels 236, 256, 276) due to the lower resistance. Once the vertical path defined by the entry regions has been filled, the respective horizontal paths (e.g., channels 236, 256, 276) may then begin to fill. In use, the channels could fill at substantially the same rate, with a slight bias to fill the bottommost channel (276) first and the topmost channel (236) last due to the slight differences in resistance. If the channels fill at slightly different rates out of order, any overflow can be collected in the vertical path defined by the exit regions (234, 254, 274). Due to the tapered exit regions, like the device 100, any air bubbles that form from differential channel filling can dissipate.

In some examples, the devices may include a layer that may act as an overflow layer alone or in a combination with any of the other features described herein, for example, shown and described in the exemplary devices shown in FIGS. 1A-2D and 7A-13D. The overflow layer can improve transduction by providing a channel in which any overflow of the testing fluid can be collected rather than be pushed into a collection device (e.g., tubing, syringe, etc.) connected to the device. This way, the cells included in the additional fluid can be transduced in the overflow channel rather than the larger diameter of the collection device. This can provide additional capacity for efficient and enhanced transduction as compared to those cells in the collection device. FIGS. 3A-6D show examples of devices including an overflow layer.

FIGS. 3A-3D show an example of a device 300 with a tapered vertical path along the exit regions like the device 100 and an overflow layer that communicates with the vertical path along the exit regions according to embodiments. As shown in the top view shown in FIG. 3A, the device 300 may have a first end 301, a second end 303, and a length therebetween. As shown in FIG. 3B, the device 300 may have a first side 305, a second side 307, and a height therebetween. As shown in FIG. 3B, the device 300 may include a plurality of stacked layers disposed vertically in parallel. In this example, the device 300 may include a (top fluid) (overflow) layer 310, a (fluid) layer 330, a (fluid) layer 350, and a (fluid) layer 370 disposed above a (bottom) layer 390.

As shown in exploded top view in FIG. 3, the layer 310 may include a first end 311, a second end 313, and a length therebetween. The layer 310 may include an overflow fluid path and an entry region 312 that corresponds to the loading region. The fluid path of the layer 310 may include a collection region 318, an exit region 314 and a channel 316 disposed there between along the length of the layer 310. The horizontal fluid path defined by the collection region 318, the exit region 314, and the channel 316, and the vertical fluid path defined by the entry region 312 can be separated on the layer so that they are not in the direct fluid communication.

The layer 330 may include a first end 331, a second end 333, and a length therebetween. The layer 330 may include a fluid path. The fluid path of the layer 330 may include an entry region 332, an exit region 334, and a channel 336 disposed there between along the length of the layer 330. The layer 330 may have a first side 335, a second side 337, and a height therebetween as shown in the cross-sectional exploded view in FIG. 3D.

Like the layer 330, the layer 350 may include a first end 351, a second end 353, and a length therebetween as shown in FIG. 3C. The layer 350 may include a fluid path. The fluid path of the layer 350 may include an entry region 352, an exit region 354, and a channel 356 disposed there between along the length of the layer 350. The layer 350 may have a first side 355, a second side 357, and a height therebetween as shown in the cross-sectional exploded view in FIG. 3D.

Like the layers 330 and 350, the layer 370 may include a first end 371, a second end 373, and a length therebetween. The layer 370 may include a fluid path. The fluid path of the layer 370 may include an entry region 372, an exit region 374, and a channel 376 disposed there between along the length of the layer 370. The layer 370 may have a first side 375, a second side 377, and a height therebetween as shown in the cross-sectional exploded view in FIG. 3D.

The bottom layer 390 may include a first end 391, a second end 393, and a length therebetween; and the layer 390 may have a first side 395, a second side 397, and a height or thickness therebetween as shown in the cross-sectional exploded view in FIG. 3D.

In this example, as shown in FIG. 3B, the layers 310, 330, 350, 370, and 390 may be stacked so that the centers of the entry regions 332, 352 and 372 are aligned with and connected to the center of the entry region/loading region 312 and the centers of the exit regions 334, 354 and 374 are aligned with and connected to the center of the exit region 314. The channels 336, 356, and 376 of the layers 330, 350, and 370, respectively may have the same channel dimensions (height, width, and length). The length of the channel 316 of the layer 310 may be smaller than the lengths of the channels 336, 356, and 376.

In this example, the layers 310, 330, 350, and 370 may also differ with respect to the width/diameter of the exit regions 314, 334, 354, and 374. As shown in FIGS. 3A-3D, like the device 100, the width/diameter of the exit region 334 may be larger than the width/diameter of the exit region 354, and the width/diameter of the exit region 354 may be larger than the width/diameter of the exit region 374. This way, the width/diameter of the exit regions can taper from larger to smaller along with the vertical path defined by the exit regions 334, 354, and 374 from the first side 305 to the second side 307.

In use, like the device 100, when loading a testing fluid into the device 300 via the loading region 312, the vertical path (regions 312, 332, 352, 372) may be filled first before the respective horizontal path (e.g., channels 336, 356, 376) due to the lower resistance. Once the vertical path defined by the entry regions 332, 352, and 372 has been filled, the respective horizontal paths (e.g., channels) may then begin to fill. In use, the channels could fill at substantially the same rate, with a slight bias to fill the bottommost channel (376) first and the topmost channel (336) last due to the slight differences in resistance. Any overflow from the vertical path defined by the exit regions (334, 354, 374) can be collected in the overflow channel 316. Due to the tapered exit regions, like the device 100, any air bubbles that could be introduced from loading and/or potentially generated from differential channel filling can dissipate. This can also prevent obstruction of the overflow channel because the air bubbles should dissipate before loading into the overflow channel begins.

FIGS. 4A-4D show an example of a device 400 with tapered vertical paths along the entry regions and the exit regions like the device 200 and an overflow layer that communicates with the vertical path along the exit regions according to embodiments. As shown in the top view shown in FIG. 4A, the device 400 may have a first end 401, a second end 403, and a length therebetween. As shown in FIG. 4B, the device 400 may have a first side 405, a second side 407, and a height therebetween. As shown in FIG. 4B, the device 400 may include a plurality of stacked layers disposed vertically in parallel. In this example, the device 400 may include a (top fluid) (overflow) layer 410, a (fluid) layer 430, a (fluid) layer 450, and a (fluid) layer 470 disposed above a (bottom) layer 490.

As shown in exploded top view in FIG. 4, the layer 410 may include a first end 411, a second end 413, and a length therebetween. The layer 410 may include an overflow fluid path and an entry region 412 that corresponds to the loading region. The fluid path of the layer 410 may include a collection region 418, an exit region 414 and a channel 416 disposed therebetween along the length of the layer 410. The horizontal fluid path defined by the collection region 418, the exit region 414, and the channel 416, and the vertical fluid path defined by the entry region 412 can be separated on the layer so that they are not in the direct fluid communication.

The layer 430 may include a first end 431, a second end 433, and a length therebetween. The layer 430 may include a fluid path. The fluid path of the layer 430 may include an entry region 432, an exit region 434, and a channel 436 disposed there between along the length of the layer 430. The layer 430 may have a first side 435, a second side 437, and a height therebetween as shown in the cross-sectional exploded view in FIG. 4D.

Like the layer 430, the layer 450 may include a first end 451, a second end 453, and a length therebetween as shown in FIG. 4C. The layer 450 may include a fluid path. The fluid path of the layer 450 may include an entry region 452, an exit region 454, and a channel 456 disposed there between along the length of the layer 450. The layer 450 may have a first side 455, a second side 457, and a height therebetween as shown in the cross-sectional exploded view in FIG. 4D.

Like the layers 430 and 450, the layer 470 may include a first end 471, a second end 473, and a length therebetween. The layer 470 may include a fluid path. The fluid path of the layer 470 may include an entry region 472, an exit region 474, and a channel 476 disposed there between along the length of the layer 470. The layer 470 may have a first side 475, a second side 477, and a height therebetween as shown in the cross-sectional exploded view in FIG. 4D.

The bottom layer 490 may include a first end 491, a second end 493, and a length therebetween; and the layer 490 may have a first side 495, a second side 497, and a height therebetween as shown in the cross-sectional exploded view in FIG. 4D.

In this example, as shown in FIG. 4B, the layers 410, 430, 450, 470, and 490 may be stacked so that the centers of the entry regions 432, 452 and 472 are aligned with and connected to the center of the entry region/loading region 412 and the centers of the exit regions 454, 454 and 474 are aligned with and connected to the center of the exit region 414. Like the device 300, the channels 436, 456, and 476 of the layers 430, 450, and 470, respectively may have the same channel dimensions (height, width, and length); and the length of the channel 416 of the layer 410 may be smaller than the lengths of the channels 436, 456, and 476.

In this example, the layers 410, 430, 450, and 470 may also differ with respect to the width/diameter of the entry regions 412, 432, 452, and 472 and the width/diameter of the exit regions 414, 434, 454, and 474. As shown in FIGS. 4A-4D, like the device 200, the width/diameter of the entry region 432 may be smaller than the width/diameter of the entry region 452, and the width/diameter of the entry region 452 may be smaller than the width/diameter of the entry region 472; and the width/diameter of the exit region 434 may be larger than the width/diameter of the exit region 454, and the width/diameter of the exit region 454 may be larger than the width/diameter of the exit region 474. This way, the width/diameter of the entry regions can taper from smaller to larger along the vertical path defined by the entry regions 432, 452, and 472 from the first side 405 to the second side 407 and the width/diameter of the exit region can taper from larger to smaller along with the vertical path defined by the exit regions 434, 454, and 474 from the first side 405 to the second side 407.

In use, like the device 300, when loading a testing fluid into the device 400 via the loading region 412, the vertical path (regions 412, 432, 452, 472) may be filled first before the respective horizontal path (e.g., channels 436, 456, 476) due to the lower resistance. Once the vertical path defined by the entry regions 432, 452, and 472 has been filled, the respective horizontal paths (e.g., channels) may then begin to fill. In use, the channels could fill at substantially the same rate, with a slight bias to fill the bottommost channel (476) first and the topmost channel (436) last due to the slight differences in resistance. Any overflow from the vertical path defined by the exit regions (434, 454, 474) can be collected in the overflow channel 416. Due to the tapered exit regions and the overflow layer, like the device 300, any air bubbles that could result from differential channel filling or during loading can dissipate before loading into the overflow channel begins and thus facilitate transduction and reduce loss of cells.

FIGS. 5A-5D show an example of a device 500 with an overflow layer that communicates with a uniform vertical path along the exit regions according to embodiments. As shown in the top view shown in FIG. 5A, the device 500 may have a first end 501, a second end 503, and a length therebetween. As shown in FIG. 5B, the device 500 may have a first side 505, a second side 507, and a height therebetween. The device 500 may include a plurality of stacked layers disposed vertically in parallel. In this example, the device 500 may include a (top fluid) (overflow) layer 510, a (fluid) layer 530, a (fluid) layer 550, and a (fluid) layer 570 disposed above a (bottom) layer 590.

As shown in exploded top view in FIG. 5C, the layer 510 may include a first end 511, a second end 513, and a length therebetween. The layer 510 may include an overflow fluid path and an entry region 512 that corresponds to the loading region. The fluid path of the layer 510 may include a collection region 518, an exit region 514 and a channel 516 disposed therebetween along the length of the layer 510. The horizontal fluid path defined by the collection region 518, the exit region 514, and the channel 516, and the vertical fluid path defined by the entry region 512 can be separated on the layer so that they are not in the direct fluid communication.

The layer 530 may include a first end 531, a second end 533, and a length therebetween. The layer 530 may include a fluid path. The fluid path of the layer 530 may include an entry region 532, an exit region 534, and a channel 536 disposed there between along the length of the layer 530. The layer 530 may have a first side 535, a second side 537, and a height therebetween as shown in the cross-sectional exploded view in FIG. 5D.

Like the layer 530, the layer 550 may include a first end 551, a second end 553, and a length therebetween as shown in FIG. 5C. The layer 550 may include a fluid path. The fluid path of the layer 550 may include an entry region 552, an exit region 554, and a channel 556 disposed there between along the length of the layer 550. The layer 550 may have a first side 555, a second side 557, and a height therebetween as shown in the cross-sectional exploded view in FIG. 5D.

Like the layers 530 and 550, the layer 570 may include a first end 571, a second end 573, and a length therebetween. The layer 570 may include a fluid path. The fluid path of the layer 570 may include an entry region 572, an exit region 574, and a channel 576 disposed there between along the length of the layer 570. The layer 570 may have a first side 575, a second side 577, and a height therebetween as shown in the cross-sectional exploded view in FIG. 5D.

The bottom layer 590 may include a first end 591, a second end 593, and a length therebetween; and the layer 590 may have a first side 595, a second side 597, and a height therebetween as shown in the cross-sectional exploded view in FIG. 5D.

In this example, as shown in FIG. 5B, the layers 510, 530, 550, 570, and 590 may be stacked so that the centers of the entry regions 532, 552 and 572 are aligned with and connected to the center of the entry region/loading region 512 and the centers of the exit regions 534, 554 and 574 are aligned with and connected to the center of the exit region 514. In this example, the entry and loading regions (512, 532, 552, and 572) have the same width/diameter; the exit regions (513, 534, 554, and 574) may have the same width/diameter; the channels 536, 556, and 576 of the layers 530, 550, and 570, respectively may have the same channel dimensions (height, width, and length); and the length of the channel 516 of the layer 510 may be smaller than the lengths of the channels 536, 556, and 576.

In use, like the devices 300 and 400, when loading a testing fluid into the device 500 via the loading region 512, the vertical path (regions 512, 532, 552, 572) may be filled first before the respective horizontal path (e.g., channels 536, 556, 576) due to the lower resistance. Once the vertical path defined by the entry regions 532, 552, and 572 has been filled, the respective horizontal paths (e.g., channels) may then begin to fill. In use, the channels could fill at substantially the same rate. Any overflow from the vertical path defined by the exit regions (534, 554, 574) can be collected in the overflow channel 516.

FIGS. 6A-6D show an example of another device 600 with an overflow layer that communicates with a uniform vertical path along the exit regions according to embodiments. In this example, the loading region of the overflow layer shown in FIGS. 5A-D can be omitted so that the entry region 632 of the layer 630 may act as the loading region.

As shown in the top view shown in FIG. 6A, the device 600 may have a first end 601, a second end 603, and a length therebetween. As shown in FIG. 6B, the device 600 may have a first side 605, a second side 607, and a height therebetween. As shown in FIG. 6B, the device 600 may include a plurality of stacked layers disposed vertically in parallel. In this example, the device 600 may include a (top fluid) (overflow) layer 610, a (fluid) layer 630, a (fluid) layer 650, and a (fluid) layer 670 disposed above a (bottom) layer 690.

As shown in exploded top view in FIG. 6, the layer 610 may include a first end 611, a second end 613, and a length therebetween. The layer 610 may include an overflow fluid path. The fluid path of the layer 610 may include a collection region 618, an exit region 614 and a channel 616 disposed therebetween along the length of the layer 610.

The layer 630 may include a first end 631, a second end 633, and a length therebetween. The layer 630 may include a fluid path. The fluid path of the layer 630 may include an entry region 632, an exit region 634, and a channel 636 disposed there between along the length of the layer 630. The layer 630 may have a first side 635, a second side 637, and a height or thickness therebetween as shown in the cross-sectional exploded view in FIG. 6D.

Like the layer 630, the layer 650 may include a first end 651, a second end 653, and a length therebetween as shown in FIG. 6C. The layer 650 may include a fluid path. The fluid path of the layer 650 may include an entry region 652, an exit region 654, and a channel 656 disposed there between along the length of the layer 650. The layer 650 may have a first side 655, a second side 657, and a height therebetween as shown in the cross-sectional exploded view in FIG. 6D.

Like the layers 630 and 650, the layer 670 may include a first end 671, a second end 673, and a length therebetween. The layer 670 may include a fluid path. The fluid path of the layer 670 may include an entry region 672, an exit region 674, and a channel 676 disposed there between along the length of the layer 670. The layer 670 may have a first side 675, a second side 677, and a height therebetween as shown in the cross-sectional exploded view in FIG. 6D.

The bottom layer 690 may include a first end 691, a second end 693, and a length therebetween; and the layer 690 may have a first side 695, a second side 697, and a height therebetween as shown in the cross-sectional exploded view in FIG. 6D.

In this example, as shown in FIG. 6B, the layers 630, 650, 670, and 690 may be stacked so that the centers of the entry regions 652 and 672 are aligned with and connected to the center of the entry region/loading region 632 and the centers of the exit regions 634, 654 and 674 are aligned with and connected to the center of the exit region 614. In this example, the entry and loading regions (632, 652, and 672) have the same width/diameter; the exit regions (614, 634, 654, and 674) may have the same width/diameter; the channels 636, 656, and 676 of the layers 630, 650, and 670, respectively may have the same channel dimensions (height, width, and length); and the length of the channel 616 of the layer 610 may be smaller than the lengths of the channels 636, 656, and 676. The length of the layer 610 may also be smaller than the layers 630, 650, 670, and 690 so that when stacked the layer 610 may not interfere with loading regions 632, 652, and 672.

In use, like the device 500, when loading a testing fluid into the device 600 via the loading region 632, the vertical path (regions 632, 652, 672) may be filled first before the respective horizontal path (e.g., channels 636, 656, 676) due to the lower resistance. Once the vertical path defined by the entry regions 632, 652, and 672 has been filled, the respective horizontal paths (e.g., channels) may then begin to fill. In use, the channels could fill at substantially the same rate. Any overflow from the vertical path defined by the exit regions (634, 654, 674) can be collected in the overflow channel 610.

In some examples, the devices may include layers having one or more different channel dimensions (e.g., width and/or length) alone or in a combination with any of the other features described herein, for example, shown and described in the exemplary devices shown in FIGS. 1A-6D and 12A-13D. The tapering channel dimensions (e.g., length and/or width) resulting in tapering surface areas or aspect ratios can improve transduction by encouraging sequential loading, which can prevent loss or heterogeneity in the cell/virus mixture throughout the channels. FIGS. 7A-11D show examples of devices having varying channel dimensions.

FIGS. 7A-D show views of a device 700 having a tapered vertical path along the exit regions of the layers and tapered length dimensions according to embodiments. As shown in the top view shown in FIG. 7A, the device 700 may have a first end 701, a second end 703, and a length therebetween. As shown in FIG. 7B, the device 700 may have a first side 705, a second side 707, and a height therebetween. As shown in FIG. 7B, the device 700 may include a plurality of stacked layers disposed vertically in parallel. In this example, the device 700 may include (top) (fluid) layer 730, a (fluid) layer 750, and a (fluid) layer 770 disposed above a (bottom) layer 790.

As shown in exploded top view in FIG. 7C, the layer 730 may include a first end 731, a second end 733, and a length therebetween. The layer 730 may include a fluid path. The fluid path of the layer 730 may include an entry region 732, an exit region 734, and a channel 736 disposed there between along the length of the layer 730. The layer 730 may have a first side 735, a second side 737, and a height therebetween as shown in the cross-sectional exploded view in FIG. 7D.

Like the layer 730, the layer 750 may include a first end 751, a second end 753, and a length therebetween as shown in FIG. 7C. The layer 750 may include a fluid path. The fluid path of the layer 750 may include an entry region 752, an exit region 754, and a channel 756 disposed there between along the length of the layer 750. The layer 750 may have a first side 755, a second side 757, and a height therebetween as shown in the cross-sectional exploded view in FIG. 7D.

Like the layers 730 and 750, the layer 770 may include a first end 771, a second end 773, and a length therebetween. The layer 770 may include a fluid path. The fluid path of the layer 770 may include an entry region 772, an exit region 774, and a channel 776 disposed there between along the length of the layer 770. The layer 770 may have a first side 775, a second side 777, and a height therebetween as shown in the cross-sectional exploded view in FIG. 7D.

The bottom layer 790 may include a first end 791, a second end 793, and a length therebetween; and a first side 795, a second side 797, and a height therebetween as shown in the cross-sectional exploded view in FIG. 7D.

In this example, the entry region 732 of the layer 730 may correspond to the loading region and the exit region 734 of the layer 730 may correspond to the collection region. As shown in FIG. 7B, the layers 730, 750, 770, and 790 may be stacked so that the centers of the entry regions 752 and 772 are aligned with and connected to the center of the entry region/loading region 732, and the exit regions 754 and 774 are connected to the respective preceding channel.

In this example, the layers 730, 750, and 770 may have channels 736, 756, 776 with the different channel dimensions (width and/or length) and entry regions 732, 752, and 776 with the same dimensions (e.g., height and width/diameter).

In this example, the layers 730, 750, and 770 may differ with respect to the length of the channels. As shown in FIGS. 7A-7D, the length of the channel 716 may be longer than the length of the channel 756, and the length of the channel 756 may be longer than the length of the channel 776.

In this example, the layers 730, 750, and 770 may also differ with respect to the width/diameter of the exit regions 734, 754, and 774. As shown in FIGS. 7A-D, the width/diameter of the exit region 734 may be larger than the width/diameter of the exit region 754, and the width/diameter of the exit region 754 may be larger than the width/diameter of the exit region 774. This way, the width/diameter of the exit region and the length of the channels of the layers can taper from larger to smaller from the first side 701 to the second side 703.

In use, when loading a testing fluid into the device 700 via the region 732, the vertical path (entry regions 732, 752, 772) may be filled first before the respective horizontal path (e.g., channels 736, 756, 776) due to the lower resistance. Once the vertical path defined by the entry regions has been filled, the respective horizontal paths (e.g., channels 736, 756, 776) may then begin to fill. In use, the channels could fill at substantially the same rate, with a slight bias to fill the bottommost channel (776) first and the topmost channel (736) last due to the slight differences in resistance and volume. Due to the tapered exit regions, any air bubbles that form from differential channel filling can dissipate.

FIGS. 8A-D show an example of a device 800 with varying channel length, like the device 700, with the same entry and exit region diameters/widths according to embodiments. As shown in the top view shown in FIG. 8A, the device 800 may have a first end 801, a second end 803, and a length therebetween. As shown in FIG. 8B, the device 800 may have a first side 805, a second side 807, and a height therebetween. The device 800 may include a plurality of stacked layers disposed vertically in parallel. In this example, the device 800 may include (top) (fluid) layer 830, a (fluid) layer 850, and a (fluid) layer 870 disposed above a (bottom) layer 890.

As shown in exploded top view in FIG. 8C, the layer 830 may include a first end 831, a second end 833, and a length therebetween. The layer 830 may include a fluid path. The fluid path of the layer 830 may include an entry region 832, an exit region 834, and a channel 836 disposed there between along the length of the layer 830. The layer 830 may have a first side 835, a second side 837, and a height therebetween as shown in the cross-sectional exploded view in FIG. 8D.

Like the layer 830, the layer 850 may include a first end 851, a second end 853, and a length therebetween as shown in FIG. 8C. The layer 850 may include a fluid path. The fluid path of the layer 850 may include an entry region 852, an exit region 854, and a channel 856 disposed there between along the length of the layer 850. The layer 850 may have a first side 855, a second side 857, and a height therebetween as shown in the cross-sectional exploded view in FIG. 8D.

Like the layers 830 and 850, the layer 870 may include a first end 871, a second end 873, and a length therebetween. The layer 870 may include a fluid path. The fluid path of the layer 870 may include an entry region 872, an exit region 874, and a channel 876 disposed there between along the length of the layer 870. The layer 870 may have a first side 875, a second side 877, and a height therebetween as shown in the cross-sectional exploded view in FIG. 8D.

The bottom layer 890 may include a first end 891, a second end 893, and a length therebetween; and a first side 895, a second side 897, and a height therebetween as shown in the cross-sectional exploded view in FIG. 8D.

In this example, the entry region 832 of the layer 830 may correspond to the loading region and the exit region 834 of the layer 830 may correspond to the collection region. As shown in FIG. 8B, the layers 830, 850, 870, and 890 may be stacked so that the centers of the entry regions 852 and 872 are aligned with and connected to the center of the entry region/loading region 832, and the exit regions 854 and 874 are connected to the respective preceding channel. The widths/diameters of the entry regions 832, 852 and 872 may be substantially the same, and the widths/diameters of the exit regions 834, 854, and 874 may be substantially the same.

In this example, the layers 830, 850, and 870 may have channels 836, 856, 876 with the different surface areas or aspect ratios. In some embodiments, the layers 830, 850, and 870 may differ with respect to the length of the channels. As shown in FIGS. 8A-8D, the length of the channel 836 may be longer than the length of the channel 856, and the length of the channel 856 may be longer than the length of the channel 876. This way, the length of the channels (i.e., surface areas or aspect ratios) of the layers can taper from larger to smaller from the first side 801 to the second side 803.

In some embodiments, each channel may have an elongated shape like those devices shown and described with respect to FIGS. 1A-8D and 10A-13D. In some embodiments, the devices may include channels having a different shape/pattern. For example, the channels, for example, as shown and described with respect to FIGS. 1A-8D and 10A-13D, may have a curvilinear or snake-like shape.

In use, when loading a testing fluid into the device 800 via the region 832, the vertical path (entry regions 832, 852, 872) may be filled first before the respective horizontal path (e.g., channels 836, 856, 876) due to the lower resistance. Once the vertical path defined by the entry regions has been filled, the respective horizontal paths (e.g., channels 836, 856, 876) may then begin to fill. In use, the channels could fill at substantially the same rate, with a slight bias to fill the bottommost channel (876) first and the topmost channel (836) last due to the slight differences in resistance and volume.

FIGS. 9A-D show an example of a device 900 with varying channel dimensions with uniform and aligned vertical paths along the entry and exit regions according to embodiments. As shown in the top view shown in FIG. 9A, the device 900 may have a first end 901, a second end 903, and a length therebetween. As shown in FIG. 9B, the device 900 may have a first side 905, a second side 907, and a height therebetween. The device 900 may include a plurality of stacked layers disposed vertically in parallel. In this example, the device 900 may include (top) (fluid) layer 930, a (fluid) layer 950, and a (fluid) layer 970 disposed above a (bottom) layer 990.

As shown in exploded top view in FIG. 9C, the layer 930 may include a first end 931, a second end 933, and a length therebetween. The layer 930 may include a fluid path. The fluid path of the layer 930 may include an entry region 932, an exit region 934, and a channel 936 disposed there between along the length of the layer 930. The layer 930 may have a first side 935, a second side 937, and a height therebetween as shown in the cross-sectional exploded view in FIG. 9D.

Like the layer 930, the layer 950 may include a first end 951, a second end 953, and a length therebetween as shown in FIG. 9C. The layer 950 may include a fluid path. The fluid path of the layer 950 may include an entry region 952, an exit region 954, and a channel 956 disposed there between along the length of the layer 950. The layer 950 may have a first side 955, a second side 957, and a height therebetween as shown in the cross-sectional exploded view in FIG. 9D.

Like the layers 930 and 950, the layer 970 may include a first end 971, a second end 973, and a length therebetween. The layer 970 may include a fluid path. The fluid path of the layer 970 may include an entry region 972, an exit region 974, and a channel 976 disposed there between along the length of the layer 970. The layer 970 may have a first side 975, a second side 977, and a height therebetween as shown in the cross-sectional exploded view in FIG. 9D.

The bottom layer 990 may include a first end 991, a second end 993, and a length therebetween; and a first side 995, a second side 997, and a height therebetween as shown in the cross-sectional exploded view in FIG. 9D.

In this example, the entry region 932 of the layer 930 may correspond to the loading region and the exit region 934 of the layer 930 may correspond to the collection region. As shown in FIG. 9B, the layers 930, 950, 970, and 990 may be stacked so that the centers of the entry regions 952 and 972 are aligned with and connected to the center of the entry region/loading region 932 and the exit regions 954 and 974 are aligned with and connected to the center of the exit region/collection region 934. The widths/diameters of the entry regions 932, 952 and 972 may be substantially the same, and the widths/diameters of the exit regions 934, 954, and 974 may be substantially the same.

In some embodiments, each channel may have two portions. For example, the channel 936 may include (straight) portions 942 that include the regions 932 and 934 and that is connected to (looped) portion 944; the channel 956 may include (straight) portions 962 that include the regions 952 and 954 and that is connected to (looped) portion 964; and the channel 976 may include (straight) portions 982 that include the regions 972 and 974 and that is connected to (looped) portion 984. In some embodiments, a device is not limited to one looped portion and may include any number of looped portions. For example, additional looped portions may be included for larger scale devices.

In this example, the layers 930, 950, and 970 may have channels 936, 956, 976 with different surface areas or aspect ratios based on difference in length. In some embodiments, the layers 930, 950, and 970 may differ with respect to the length of the channels. As shown in FIGS. 9A-9D, the overall length of the channel 936 may be longer than the overall length of the channel 956, and the overall length of the channel 956 may be longer than the overall length of the channel 976. The length of channels may taper with respect to portion 944 of the channel 936, with respect to the portion 964 of the channel 956, and with respect to the portion 984 of the channel 976. The length of the portions 942 of the channel 936, the portion 962 of the channel 956, and the portion 82 of the channel 976 may be substantially the same. This way, the overall length of the channels of the layers can taper from larger to smaller from the first side 901 to the second side 903 while maintaining the alignments of the entry regions 932, 952, and 972 and exit regions 934, 954 and 974.

In use, when loading a testing fluid into the device 900 via the region 932, the vertical path (entry regions 932, 952, 972) may be filled first before the respective horizontal path (e.g., channels 936, 956, 976) due to the lower resistance. Once the vertical path defined by the entry regions has been filled, the respective horizontal paths (e.g., channels 936, 956, 976) may then begin to fill. In use, the channels could fill at substantially the same rate, with a slight bias to fill the bottommost channel (976) first and the topmost channel (936) last due to the slight differences in resistance and volume. If the channels fill at slightly different rates out of order, any overflow can be collected in the vertical path defined by the exit regions (934, 954, 974).

In some embodiments, other channel dimension(s), such as width of the channels, may also be varied so that the surface area or aspect ratio of the channels vary between layers. FIGS. 10A-D show views of a device 1000 having a tapered vertical path along the exit regions of the layers and a taper along vertical path of the exit regions according to embodiments. As shown in the top view shown in FIG. 10A, the device 1000 may have a first end 1001, a second end 1003, and a length therebetween. The device 1000 may also have a first side 1002, a second side 1004, and a width therebetween. As shown in FIG. 10B, the device 1000 may have a first side 1005, a second side 1007, and a height therebetween. As shown in FIG. 10B, the device 1000 may include a plurality of stacked layers disposed vertically in parallel. In this example, the device 1000 may include (top) (fluid) layer 1030, a (fluid) layer 1050, and a (fluid) layer 1070 disposed above a (bottom) layer 1090.

As shown in exploded top view in FIG. 10C, the layer 1030 may include a first end 1031, a second end 1033, and a length therebetween. The layer 1030 may include a first side 1042, a second side 1044, and a width therebetween. The layer 1030 may include a fluid path. The fluid path of the layer 130 may include an entry region 1032, an exit region 1034, and a channel 1036 disposed there between along the length of the layer 1030. The layer 1030 may have a first side 1035, a second side 1037, and a height therebetween as shown in the cross-sectional exploded view in FIG. 10D.

Like the layer 1030, the layer 1050 may include a first end 1051, a second end 1053, and a length therebetween as shown in FIG. 10C. The layer 1050 may include a first side 1062, a second side 1064, and a width therebetween. The layer 1050 may include a fluid path. The fluid path of the layer 1050 may include an entry region 1052, an exit region 1054, and a channel 1056 disposed there between along the length of the layer 1050. The layer 1050 may have a first side 1055, a second side 1057, and a height therebetween as shown in the cross-sectional exploded view in FIG. 10D.

Like the layers 1030 and 1050, the layer 1070 may include a first end 1071, a second end 1073, and a length therebetween. The layer 1070 may include a first side 1082, a second side 1084, and a width therebetween. The layer 1070 may include a fluid path. The fluid path of the layer 1070 may include an entry region 1072, an exit region 1074, and a channel 1076 disposed there between along the length of the layer 1070. The layer 1070 may have a first side 1075, a second side 1077, and a height therebetween as shown in the cross-sectional exploded view in FIG. 10D.

The bottom layer 1090 may include a first end 1091, a second end 1093, and a length therebetween; and a first side 1095, a second side 1097, and a height therebetween as shown in the cross-sectional exploded view in FIG. 10D.

In this example, the entry region 1032 of the layer 1030 may correspond to the loading region and the exit region 1034 of the layer 1030 may correspond to the collection region. As shown in FIG. 10B, the layers 1030, 1050, 1070, and 1090 may be stacked so that the centers of the entry regions 1052 and 1072 are aligned with and connected to the center of the entry region/loading region 1032 and the centers of the exit regions 1054 and 1074 are aligned and connected to the center of the exit region/collection region 1034.

In this example, the layers 1030, 1050, and 1070 may have channels 1036, 1056, 1076 with different channel dimensions (e.g., width with respect to 1002/1004) and entry regions 1032, 1052, and 1072 that have the same dimensions (e.g., height and width/diameter). For example, each channel 1036, 1056, and 1076 of the layers 1030, 1050, and 1070 may have a width that gradually increases and decreases along its length between the respective entry and exit regions. As shown in FIGS. 10A and 10C, the surface area or aspect ratio based on the graduation of widths of the channel 1036 may be larger than the surface area or aspect ratio based on the graduation of widths of the channel 1056, and the surface area or aspect ratio based on the graduation of widths of the channel 1056 may be larger than the surface area or aspect ratio based on the graduation of widths of the channel 1076. This way, the widths of the channels 1036, 1056, 1076 of the layers 1030, 1050, 1070 can taper from larger to smaller from the first side 1005 to the second side 1007 of the device 1000.

In this example, the layers 1030, 1050, and 1070 may also differ with respect to the width/diameter of the exit regions 1034, 1054, and 1074. As shown in FIGS. 10A-D, the width/diameter of the exit region 1034 may be larger than the width/diameter of the exit region 1054, and the width/diameter of the exit region 1054 may be larger than the width/diameter of the exit region 1074. This way, the width/diameter of the exit region 1034, 1054, 1074 and the widths of the channels 1036, 1056, 1076 of the layers 1030, 1050, 1070 can taper from larger to smaller from the first side 1005 to the second side 1007.

In use, when loading a testing fluid into the device 1000 via the region 1032, the vertical path (entry regions 1032, 1052, 1072) may be filled first before the respective horizontal path (e.g., channels 1036, 1056, 1076) due to the lower resistance. Once the vertical path defined by the entry regions has been filled, the respective horizontal paths (e.g., channels 1036, 1056, 1076) may then begin to fill. In use, the channels could fill at substantially the same rate, with a slight bias to fill the bottommost channel (1076) first and the topmost channel (1036) last due to the slight differences in resistance and volume. If the channels fill at slightly different rates out of order, any overflow can be collected in the vertical path defined by the exit regions (1034, 1054, 1074).

FIGS. 11A-D show an example of a device 1100 with tapering surface area or aspect ratio based on the graduations of widths within each channel with uniform vertical paths along the entry and exit regions according to embodiments. As shown in the top view shown in FIG. 11A, the device 1100 may have a first end 1101, a second end 1103, and a length therebetween. As shown in FIG. 11B, the device 1100 may have a first side 1105, a second side 1107, and a height therebetween. The device 1100 may include a plurality of stacked layers disposed vertically in parallel. In this example, the device 1100 may include (top) (fluid) layer 1130, a (fluid) layer 1150, and a (fluid) layer 1170 disposed above a (bottom) layer 1190.

As shown in exploded top view in FIG. 11C, the layer 1130 may include a first end 1131, a second end 1133, and a length therebetween. The layer 1130 may include a fluid path. The fluid path of the layer 1130 may include an entry region 1132, an exit region 1134, and a channel 1136 disposed there between along the length of the layer 1130. The layer 1130 may have a first side 1135, a second side 1137, and a height therebetween as shown in the cross-sectional exploded view in FIG. 11D.

Like the layer 1130, the layer 1150 may include a first end 1151, a second end 1153, and a length therebetween as shown in FIG. 11C. The layer 1150 may include a fluid path. The fluid path of the layer 1150 may include an entry region 1152, an exit region 1154, and a channel 1156 disposed there between along the length of the layer 1150. The layer 1150 may have a first side 1155, a second side 1157, and a height therebetween as shown in the cross-sectional exploded view in FIG. 11D.

Like the layers 1130 and 1150, the layer 1170 may include a first end 1171, a second end 1173, and a length therebetween. The layer 1170 may include a fluid path. The fluid path of the layer 1170 may include an entry region 1172, an exit region 1174, and a channel 1176 disposed there between along the length of the layer 1170. The layer 1170 may have a first side 1175, a second side 1177, and a height therebetween as shown in the cross-sectional exploded view in FIG. 11D.

The bottom layer 1190 may include a first end 1191, a second end 1193, and a length therebetween; and a first side 1195, a second side 1197, and a height therebetween as shown in the cross-sectional exploded view in FIG. 11D.

In this example, the entry region 1132 of the layer 1130 may correspond to the loading region and the exit region 1134 of the layer 1130 may correspond to the collection region. As shown in FIG. 11B, the layers 1130, 1150, 1170, and 1190 may be stacked so that the centers of the entry regions 1152 and 1172 are aligned with and connected to the center of the entry region/loading region 1132, and the exit regions 1154 and 1174 are connected to the respective preceding channel. The widths/diameters of the entry regions 1132, 1152 and 1172 may be substantially the same, and the widths/diameters of the exit regions 1134, 1154, and 1174 may be substantially the same.

In this example, the layers 1130, 1150, and 1170 may have channels 1136, 1156, 1176 with different channel dimensions (e.g., width with respect to 1102/1104) and regions 1132, 1134, 1152, 1154, 1172, 1174 with the same region dimensions. In some embodiments, the channels 1136, 1156, 1176 of the layers 1130, 1150, and 1170 may differ with respect to the surface area or aspect ratio based on the different widths of the respective channel. For example, each channel 1136, 1156, and 1176 of the layers 1130, 1150, and 1170 may have a width that gradually increases and decreases along its length between the respective entry and exit regions. As shown in FIGS. 11A and 11C, the surface area or aspect ratio based on the graduation of widths of the channel 1136 may be larger than the surface area or aspect ratio based on the graduation of widths of the channel 1156, and the surface area or aspect ratio based on the graduation of widths of the channel 1156 may be larger than the surface area or aspect ratio based on the graduation of widths of the channel 1176. This way, the widths of the channels 1136, 1156, 1176 of the layers 1130, 1150, 1170 can taper from larger to smaller from the first side 1105 to the second side 1107.

In use, when loading a testing fluid into the device 1100 via the region 1132, the vertical path (entry regions 1132, 1152, 1172) may be filled first before the respective horizontal path (e.g., channels 1136, 1156, 1176) due to the lower resistance. Once the vertical path defined by the entry regions has been filled, the respective horizontal paths (e.g., channels 1136, 1156, 1176) may then begin to fill. In use, the channels could fill at substantially the same rate, with a slight bias to fill the bottommost channel (1176) first and the topmost channel (1136) last due to the slight differences in resistance and volume. If the channels fill at slightly different rates out of order, any overflow can be collected in the vertical path defined by the exit regions (1134, 1154, 1174). Due to the tapered exit regions, any air bubbles that form from differential channel filling can dissipate.

In some examples, the devices may include features configured for bottom loading of the device, alone or in a combination with any of the other features described herein, for example, shown and described in the exemplary devices shown in FIGS. 1A-11D. By configuring the channels such that the testing fluid enters from the loading region directly into the entry region of the channel closest to the bottom layer, an ordered loading from bottom to top can be achieved. FIGS. 12A-13D show examples of devices configured for bottom-loading.

FIGS. 12A-D show views of a device 1200 having a tapered vertical path along the exit regions of the layers and configured for bottom-loading according to embodiments. As shown in the top view shown in FIG. 12A, the device 1200 may have a first end 1201, a second end 1203, and a length therebetween. As shown in FIG. 12B, the device 1200 may have a first side 1205, a second side 1207, and a height therebetween. As shown in FIG. 12B, the device 1200 may include a plurality of stacked layers disposed vertically in parallel. In this example, the device 1200 may include (top) (fluid) layer 1230, a (fluid) layer 1250, and a (fluid) layer 1270 disposed above a (bottom) layer 1290.

In this example, each fluid layer may include a fluid path disposed horizontally with respect to the length of the device. Each fluid layer may include a loading region, an entry region, an exit region, and a channel that at least extends between the entry region and the exit region disposed horizontally with respect to the device length.

As shown in exploded top view in FIG. 12C, the layer 1230 may include a first end 1231, a second end 1233, and a length therebetween. The layer 1230 may include a fluid path and a loading region 1238. The fluid path of the layer 1230 may include an entry region 1232, an exit region 1234, and a channel 1236 disposed there between along the length of the layer 1230. The layer 1230 may have a first side 1235, a second side 1237, and a height therebetween as shown in the cross-sectional exploded view in FIG. 12D.

Like the layer 1230, the layer 1250 may include a first end 1251, a second end 1253, and a length therebetween as shown in FIG. 1C. The layer 1250 may include a fluid path and a loading region 1258. The fluid path of the layer 1250 may include an entry region 1252, an exit region 1254, and a channel 1256 disposed there between along the length of the layer 1250. The layer 1250 may have a first side 1255, a second side 1257, and a height therebetween as shown in the cross-sectional exploded view in FIG. 12D.

Like the layers 1230 and 1250, the layer 1270 may include a first end 1271, a second end 1273, and a length therebetween. The layer 1270 may include a fluid path. The fluid path of the layer 1270 may include a loading region 1278, an entry region 1272, an exit region 1274, and a channel 1276 disposed between the loading region 1278 and the exit region 1274 along the length of the layer 1270. The layer 1270 may have a first side 1275, a second side 1277, and a height therebetween as shown in the cross-sectional exploded view in FIG. 1D.

The bottom layer 1290 may include a first end 1291, a second end 1293, and a length therebetween; and a first side 1295, a second side 1297, and a height therebetween as shown in the cross-sectional exploded view in FIG. 12D.

In this example, the loading region 1238 of the layer 1230 may correspond to the loading region that receives the testing fluid and the exit region 1234 of the layer 1230 may correspond to the collection region. As shown in FIG. 12B, the layers 1230, 1250, 1270, and 1290 may be stacked so that (i) the centers of the loading regions 1258 and 1278 are aligned with and connected to the center of the loading region 1238; (ii) the centers of the entry regions 1252 and 1272 are aligned with and connected to the center of the entry region/loading region 1232; and (iii) the centers of the exit regions 1254 and 1274 are aligned with and connected to the center of the exit region/collection region 1234. This way, the device 1200 may include three vertical paths. The layers 1230 and 1250 may have channels 1236 and 1256 with the same channel dimensions (height, width, and length) and entry regions 1232, 1252, and 1276 with the same dimensions (e.g., height and width/diameter).

In some embodiments, the length of the channel 1276 may be longer than the channels 1236 and 1256. This way the vertical path of the loading regions (1238, 1258, and 1278) may directly connect to the channel 1276.

In this example, the layers 1230, 1250, and 1270 may differ with respect to the width/diameter of the exit regions 1234, 1254, and 1274. As shown in FIGS. 12A-12D, the width/diameter of the exit region 1234 may be larger than the width/diameter of the exit region 1254, and the width/diameter of the exit region 1254 may be larger than the width/diameter of the exit region 1274. This way, the width/diameter of the exit region tapers from larger to smaller along with the vertical path defined by the exit regions 1234, 1254, and 1274 from the first side 1201 to the second side 1203.

In use, when loading a testing fluid into the device 1200 via the region 1238, the vertical path (regions 1238, 1258, 1278) and the bottom channel 1276 may be filled first before entering the paths defined by the respective horizontal path (e.g., channels 1236 and 1256) due to the lower resistance. Once the vertical path defined by the loading regions has been filled, the channel 1276 and the vertical path defined by the regions 1232 and 1252 may begin to fill via the region 1272. After which, the respective horizontal paths (e.g., channels) may then begin to fill. In use, the channels could fill at substantially the same rate, with a slight bias to fill the bottommost channel (1276) first and the topmost channel (1236) last due to the slight differences in resistance. If the channels fill at slightly different rates out of order, any overflow can be collected in the vertical path defined by the exit regions (1234, 1254, 1274). Due to the tapered exit regions, any air bubbles that form from differential channel filling can dissipate. The decreasing exit region diameter can prevent air bubbles from getting trapped.

FIGS. 13A-D show an example of a device 1300 with uniform vertical paths along the regions and configured to bottom-load according to embodiments. FIGS. 13A-D show views of the device 1300. As shown in the top view shown in FIG. 13A, the device 1300 may have a first end 1301, a second end 1303, and a length therebetween. As shown in FIG. 13B, the device 1300 may have a first side 1305, a second side 1307, and a height therebetween. As shown in FIG. 13B, the device 1300 may include a plurality of stacked layers disposed vertically in parallel. In this example, the device 1300 may include (top) (fluid) layer 1330, a (fluid) layer 1350, and a (fluid) layer 1370 disposed above a (bottom) layer 1390.

In this example, each fluid layer may include a fluid path disposed horizontally with respect to the length of the device. Each fluid layer may include a loading region, an entry region, an exit region, and a channel that extends at least between the entry region and the exit region disposed horizontally with respect to the device length.

As shown in exploded top view in FIG. 13C, the layer 1330 may include a first end 1331, a second end 1333, and a length therebetween. The layer 1330 may include a fluid path and a loading region 1338. The fluid path of the layer 1330 may include an entry region 1332, an exit region 1334, and a channel 1336 disposed there between along the length of the layer 1330. The layer 1330 may have a first side 1335, a second side 1337, and a height therebetween as shown in the cross-sectional exploded view in FIG. 13D.

Like the layer 1330, the layer 1350 may include a first end 1351, a second end 1353, and a length therebetween as shown in FIG. 13C. The layer 1350 may include a fluid path and a loading region 1358. The fluid path of the layer 1350 may include an entry region 1352, an exit region 1354, and a channel 1356 disposed there between along the length of the layer 1350. The layer 1350 may have a first side 1355, a second side 1357, and a height therebetween as shown in the cross-sectional exploded view in FIG. 13D.

Like the layers 1330 and 1350, the layer 1370 may include a first end 1371, a second end 1373, and a length therebetween. The layer 1370 may include a fluid path. The fluid path of the layer 1370 may include a loading region 1378, an entry region 1372, an exit region 1374, and a channel 1376 disposed between the loading region 1378 and the exit region 1374 along the length of the layer 1370. The layer 1370 may have a first side 1375, a second side 1377, and a height therebetween as shown in the cross-sectional exploded view in FIG. 13D.

The bottom layer 1390 may include a first end 1391, a second end 1393, and a length therebetween; and a first side 1395, a second side 1397, and a height therebetween as shown in the cross-sectional exploded view in FIG. 13D.

In this example, the loading region 1338 of the layer 1330 may correspond to the loading region that receives the testing fluid and the exit region 1334 of the layer 1330 may correspond to the collection region. As shown in FIG. 13B, the layers 1330, 1350, 1370, and 1390 may be stacked so that (i) the centers of the loading regions 1358 and 1378 are aligned with and connected to the center of the loading region 1338; (ii) the centers of the entry regions 1352 and 1372 are aligned with and connected to the center of the entry region/loading region 1332; and (iii) the centers of the exit regions 1354 and 1374 are aligned with and connected to the center of the exit region/collection region 1334. This way, the device 1300 may include three vertical paths.

As shown in FIGS. 13A-D, the layers 1330 and 1350 may have channels 1336 and 1356 with the same channel dimensions (height, width, and length). The layers 1330, 1350 and 1370 may have loading regions 1338, 1358, and 1378 may have the same dimensions (e.g., height and width/diameter); the entry regions 1332, 1352, and 1372 may have the same dimensions (e.g., height and width/diameter); and the exit regions 1336, 1356, and 1376 may have the same dimensions (height and width/diameter).

In some embodiments, the length of the channel 1376 may be longer than the channels 1336 and 1356. This way, the vertical path of the loading regions (1338, 1358, and 1378) may directly connect to the channel 1376.

In use, when loading a testing fluid into the device 1330 via the region 1338, the vertical path (regions 1338, 1358, 1378) and the bottom channel 1376 may be filled first before entering the paths defined by the respective horizontal path (e.g., channels 1336 and 1356). Once the vertical path defined by the loading regions has been filled, the channel 1376 and the vertical path defined by the regions 1332 and 1352 may begin to fill via the region 1372. After which, the respective horizontal paths (e.g., channels) may then begin to fill. In use, the channels could fill at substantially the same rate, with a slight bias to fill the bottommost channel (1376) first and the topmost channel (1336) last. If the channels fill at slightly different rates out of order, any overflow can be collected in the vertical path defined by the exit regions (1334, 1354, 1374).

In some embodiments, the devices may be made of any material, including but not limited to, fluorinated ethylene propylene (FEP), polymethyl methacrylate (PMMA), cyclic olefin polymer (COP), cyclic olefin copolymer (COC), UV curable resins (such as glass (e.g., Pyrex), silicon and polymers (e.g., polydimethylsiloxane (PDMS), polystyrene (PS), polycarbonate (PC) or polyvinyl chloride (PVC)), other gas permeable/liquid impermeable materials, among others, or any combination thereof.

In some embodiments, the layers of the devices can be laminated together with or without adhesives to form the devices shown and described in the figures using any known technology. For example, the layers of the device can also be held together using thread forming screws, nuts and bolts, clips, clamps, pins, alignment holes/pegs, ultrasonic welding, solvent-assisted bonding, heat staking, thermal bonding, laser welding, snap fits, glue (e.g., biocompatible, low absorption adhesives such as acrylates) and/or surface treatment (e.g., oxygen plasma). During the assembly, a microscope or other machinery can be used to assist with the alignment of the components.

By way of example, one or more of the (fluid) layers may include a biocompatible material (e.g., FEP) having adhesive on each side so that it may be disposed between two substrates, such as PDMS slabs, other FEP sheets, or a combination thereof.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims.

1. A device comprising:
two or more stacked layers, the two or more stacked layers including a first layer and a second layer;
the first layer including a first fluid path, the first fluid path including a first entry region having a first entry region diameter, a first exit region having a first exit region diameter, and a first channel disposed between the first entry region and the first exit region, the first channel having first channel dimensions, the first channel dimensions including a first channel length and a first channel width;
the second layer including a second fluid path that is parallel to the first fluid path and is in fluid communication with the first fluid path, the second fluid path including a second entry region having a second entry region diameter, a second exit region having a second exit region diameter, and a second channel disposed between the second entry region and the second exit region, the second channel having second channel dimensions; the second channel dimensions including a second channel length and a second channel width;
wherein (i) the first entry region diameter and the second entry region diameter are different; and/or (ii) the first exit region diameter and the second exit region diameter are different; and/or (iii) one or more of the first channel dimensions and the one or more of the second channel dimensions are different.

2. The device of claim 1, wherein the first entry region and the second entry region are aligned with respect to its respective center and are in fluid communication.

3. The device of claim 1, wherein the first exit region and the second exit region are aligned with respect to its respective center and are in fluid communication:

4. The device of claim 3, wherein:
the first exit region diameter is larger than the second exit region diameter.

5. The device of claim 3, wherein:
the first entry region diameter is smaller than the second entry region diameter.

6. The device of claim 1, the device further comprising:
a third layer including at least one loading region and/or at least one collection region;
wherein the first layer is disposed between the third layer and the second layer.

7. The device of claim 6, wherein:
the at least one loading region of the third layer is aligned with the first entry region and the second entry region with respect to its respective centers and are in fluid communication.

8. The device of claim 7, wherein:
the third layer includes a fluid path, the fluid path including the at least one collection outlet, an exit region and a channel disposed therebetween;
the exit region of the third layer is aligned with the first exit region and the second exit region with respect to its respective centers and are in fluid communication.

9. The device of claim 8, wherein the at least one loading region of the third layer is separate from the fluid path of the third layer.

10. The device of claim 9, wherein:
each layer includes at least one loading region;
the at least one loading region of the first layer is separate from the first fluid path; and
the at least one loading region of the second layer is in fluid communication with the second fluid path; and
the at least one loading region of the first layer and the at least loading region of the second layer are aligned with respect to its respective centers and are in fluid communication.

11. A device comprising:
two or more layers, the two or more stacked layers including a first layer and a second layer;
the first layer including a first fluid path, the first fluid path including a first entry region having a first entry region diameter, a first exit region having a first exit region diameter, and a first channel disposed between the first entry region and the first exit region, the first channel having first channel dimensions, the first channel dimensions including a first channel length and a first channel width;
the second layer including a second fluid path that is in fluid communication with the first fluid path, the second fluid path including a second entry region having a second entry region diameter, a second exit region having a second exit region diameter, and second channel disposed between the second entry region and the second exit region, the second channel having a second channel dimensions; the second channel dimensions including a second channel length and a second channel width;
wherein the first exit region diameter and the second exit region diameter are different.

12. The device of claim 11, wherein the first exit region diameter is larger than the second exit region diameter.

13. The device of claim 11, wherein the first exit region and the second exit region are aligned with respect to its respective center and are in fluid communication:

14. The device of claim 13, wherein:
the first entry region diameter is smaller than the second entry region diameter.

15. The device of claim 13, the device further comprising:
a third layer including at least one loading region and/or at least one collection region;
wherein the first layer is disposed between the third layer and the second layer.

16. The device of claim 15, wherein the at least one loading region of the third layer is aligned with the first entry region and the second entry region with respect to its respective centers and are in fluid communication.

17. The device of claim 16 wherein:
the third layer includes a fluid path, the fluid path including the at least one collection region, an exit region, and a channel disposed therebetween;
the exit region of the third layer is aligned with the first exit region and the second exit region with respect to its respective centers and are in fluid communication.

18. The device of claim 17, wherein:
the at least one loading region is separate from the fluid path;
the at least one loading region is aligned with the first entry region and the second entry region with respect to its respective centers and are in fluid communication.

19. The device of any of claim 17, wherein:
each layer includes at least one loading region.

20. The device of claim 19, wherein:
the at least one loading region of the first layer is separate from the fluid communication with the first fluid path;
the at least one loading region of the second layer is in fluid communication with the second fluid path; and
the at least one loading region of the first region and the at least loading region of the second layer are aligned with respect to its respective centers and are in fluid communication.
